(19) United States
(12) Patent Application Publication
(10) Pub. No.: US 2021/0205224 A1
(43) Pub. Date: Jul. 8, 2021

(54) ELECTROSPRAYED AND ELECTROSPUN CANNABINOID COMPOSITIONS AND PROCESS TO PRODUCE

(71) Applicant: Cannabis Global, Inc., Los Angeles, CA (US)

(72) Inventor: Joseph Noel, Seal Beach, CA (US)

(21) Appl. No.: 17/089,497

(22) Filed: Nov. 4, 2020

Related U.S. Application Data
(60) Provisional application No. 62/930,358, filed on Nov. 4, 2019.

(51) Int. Cl. A61K 9/16; A61K 31/047; A61K 31/352

(52) U.S. Cl. CPC A61K 9/1658; A61K 31/352; A61K 31/047

(57) ABSTRACT

A composition comprising a plurality of discrete particles comprising one or more cannabinoids disposed at least partially within a polymeric carrier having a maximum overall dimension of less than 1 micron. A composition comprising a plurality of discrete nanofibers comprising one or more cannabinoids disposed at least partially within a polymeric carrier is also disclosed along with methods to produce the same.

### RELATED APPLICATIONS

This application claims the benefit of a U.S. Provisional Application Ser. No. 62/930,358 filed Nov. 4, 2019, the disclosure of which is incorporated by reference herein in its entirety.

### FIELD

This disclosure relates to a composition comprising a plurality of discrete particles and/or a plurality of nanofibers comprising one or more cannabinoids disposed at least partially within a water soluble or water miscible carrier.

### BACKGROUND

Delivery of biologically active compounds such as pharmaceuticals and so-called nutraceuticals to living systems is the object of much study and research. Making a material available to a living system may be further complicated by the solubility profile of the material. While technologies exist, which may render a material with low water solubility available to a living system, such technologies typically have other drawbacks rendering their use limited to particular instances.

One attractive means of delivering biologically active material to living organisms, such as human beings, is via an ingestible carrier. The art is replete with such systems in which the active material is dissolved or otherwise emulsified within a substrate, and the substrate is then eaten or dissolved in a liquid and consumed by the end user. Edible or otherwise consumable films may be adapted to be water and/or mucosally dissolvable and then swallowed by an end user. US 2004/0247647 is generally directed to a breath freshening film adapted to rapidly dissolve in the mouth of a consumer comprising a high viscosity and a low viscosity film forming agent such as hydroxypropyl methylcellulose (HPMC) for improved strength during processing and storage. Other references include US20170127711, which is generally directed to a water soluble package comprising HPMC, along with other materials to render a hydrophobic component ingestible.

However, providing a means to allow ingestion of a biologically active component does not necessarily render that component biologically available to the host. This is especially true with hydrophobic materials, i.e., have limited water solubility. There remains a need in the art to provide biologically active materials to a living system. Further, a need exists to render various dosages with accuracy and precision.

### SUMMARY

In one aspect of the disclosure, a composition comprises a plurality of discrete particles comprising one or more cannabinoids disposed at least partially within a polymeric carrier having a maximum overall dimension of less than 1 micron.

In another aspect of this disclosure, a composition comprises a plurality of discrete nanofibers comprising one or more cannabinoids disposed at least partially within a polymeric carrier having a maximum width of less than 1 micron.

In one aspect of the disclosure, a process to produce a composition comprises the steps of: a) providing one or more precursor mixtures comprising one or more cannabinoids and one or more polymeric carrier components in a solvent; and

i) electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles; or

ii) electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.

### BRIEF DESCRIPTION OF THE DRAWINGS

FIG. 1a is a micrograph showing gelatin nanofibers containing CBD at 5,000× according to embodiments of the disclosure;

FIG. 1b is a micrograph showing gelatin nanofibers containing CBD at 10,000× according to embodiments of the disclosure;

FIG. 2a is a micrograph showing comparative nanofibers at 5,000× according to embodiments of the disclosure;

FIG. 2b is a micrograph showing comparative nanofibers at 10,000× according to embodiments of the disclosure;

FIG. 3a is a micrograph showing THC containing particles produced from PCL50 at 2,500× according to embodiments of the disclosure;

FIG. 3b is a micrograph showing THC containing particles produced from PCL50 at 2,500× according to embodiments of the disclosure;

FIG. 3c is a micrograph showing THC containing particles produced from PCL50 at 2,500× according to embodiments of the disclosure;

FIG. 3d is a micrograph showing THC containing particles produced from PCL50 at 2,500× according to embodiments of the disclosure;

FIG. 4a is a micrograph showing comparative nanoscale particles produced from PCL50 at 10,000× according to embodiments of the disclosure;

FIG. 4b is a micrograph showing comparative nanoscale particles produced from PCL50 at 10,000× according to embodiments of the disclosure;

FIG. 4c is a micrograph showing comparative nanoscale particles produced from PCL50 at 10,000× according to embodiments of the disclosure;

FIG. 5a is a micrograph showing nanoscale particles containing CBD, produced from PCL50 at 2,500× according to embodiments of the disclosure;

FIG. 5b is a micrograph showing a CBD/PCL50 nanoscale particle shown in FIG. 5a at 10,000× according to embodiments of the disclosure;

FIG. 5c shows the particles of FIG. 5a dispersed in water;

FIG. 6a is a micrograph showing nanoscale particles containing CBD at 10,000× according to embodiments of the disclosure;

FIG. 6b is a micrograph showing nanoscale particles containing CBD at 20,000× according to embodiments of the disclosure;

FIG. 7a is a micrograph showing comparative nanoscale particles at 10,000× according to embodiments of the disclosure;

FIG. 7b is a micrograph showing nanoscale fibers containing CBD at 20,000× according to embodiments of the disclosure;

FIG. 7c is a micrograph showing nanoscale fibers containing CBD at 10,000× according to embodiments of the disclosure;

FIG. 8a is a micrograph showing nanoscale particles containing CBD at 2,500× according to embodiments of the disclosure;

FIG. 8b is a micrograph showing nanoscale particles containing CBD at 20,000× according to embodiments of the disclosure;

FIG. 9a shows an agglomerate of nanoscale particles containing CBD according to embodiments of the disclosure;

FIG. 9b shows the particles of FIG. 9a in a drop of water;

FIG. 9c shows the same as FIG. 9b after 5 minutes at room temperature;

FIG. 9d shows an agglomerate of nanoscale particles containing CBD according to embodiments of the disclosure;

FIG. 9e shows the particles of FIG. 9d in a drop of water; and

FIG. 9f shows the same as FIG. 9e after 5 minutes at room temperature.

FIG. 10a is and electron micrograph showing a plurality of electrospun nanofibers according to an embodiment of the disclosure;

FIG. 10b is and electron micrograph showing a plurality of electrospun nanofibers according to another embodiment of the disclosure;

FIG. 11a is and electron micrograph showing a plurality of electrospun nanofibers produced by coaxial electrospinning according to an embodiment of the disclosure;

FIG. 11b is and electron micrograph showing a plurality of electrospun nanofibers produced by coaxial electrospinning according to another embodiment of the disclosure;

FIG. 12a is histogram showing fibermatic analysis of the nanofibers shown in FIG. 12e produced by electrospinning according to another embodiment of the disclosure;

FIG. 12b is histogram showing fibermatic analysis of the nanofibers shown in FIG. 12f produced by electrospinning according to another embodiment of the disclosure;

FIG. 12c is histogram showing fibermatic analysis of the nanofibers shown in FIG. 12g produced by electrospinning according to another embodiment of the disclosure;

FIG. 12d is histogram showing fibermatic analysis of the nanofibers shown in FIG. 12h produced by electrospinning according to another embodiment of the disclosure;

FIG. 12e is and electron micrograph showing a plurality of electrospun nanofibers produced by coaxial electrospinning according to an embodiment of the disclosure;

FIG. 12f is and electron micrograph showing a plurality of electrospun nanofibers produced by coaxial electrospinning according to an embodiment of the disclosure;

FIG. 12g is and electron micrograph showing a plurality of electrospun nanofibers produced by coaxial electrospinning according to an embodiment of the disclosure; and

FIG. 12h is and electron micrograph showing a plurality of electrospun nanofibers produced by coaxial electrospinning according to an embodiment of the disclosure.

### DETAILED DESCRIPTION

Bioavailability of cannabinoids is problematic due to the lack of water solubility of these materials. Bioavailability may be increase by reducing the size of the discrete particles or droplets of these materials in a composition. Electrospraying carrier materials along with cannabinoids allows for the formation of compositions including nanosized particles which include nanosized amounts of cannabinoids and thus increasing bioavailability upon ingestion of these compositions. The same is true for electrospinning of these materials, which produces nanosized fibers. These compositions may have in excess of 30 wt % cannabinoids and may be produced using water soluble carriers. The end result is compositions including cannabinoids which are water soluble.

### Definitions

For the purposes of this disclosure and the claims thereto, the new numbering scheme for the Periodic Table Groups is used as described in CHEMICAL AND ENGINEERING NEWS, 63(5), p. 27 (1985). Therefore, a “Group 4 metal” is an element from Group 4 of the Periodic Table.

A “homopolymer” is a polymer having mer units that are the same. A “copolymer” is a polymer having two or more mer units that are different from each other. A “terpolymer” is a polymer having three mer units that are different from each other. “Different” as used to refer to mer units indicates that the mer units differ from each other by at least one atom or are different isomerically. Accordingly, the definition of copolymer, as used herein, includes terpolymers and the like. An “cellulosic polymer” or “cellulosic copolymer” is a polymer or copolymer comprising at least 50 mol % cellulose derived units.

For purposes of this disclosure and claims thereto, the term “substituted” means that a hydrogen group has been replaced with a hydrocarbyl group, a heteroatom, or a heteroatom containing group. For example, a “substituted hydrocarbyl” is a radical made of carbon and hydrogen where at least one hydrogen is replaced by an alkyl group, a heteroatom or heteroatom containing group. The terms “hydrocarbyl radical,” “hydrocarbyl group,” or “hydrocarbyl” may be used interchangeably and are defined to mean a group consisting of hydrogen and carbon atoms only. Preferred hydrocarbyls are C₁-C₁₀₀ radicals that may be linear, branched, or cyclic, and when cyclic, aromatic or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl naphthyl, and the like.

Unless otherwise indicated, (e.g., the definition of “substituted hydrocarbyl”, “substituted cannabinol,” etc.), the term “substituted” means that at least one hydrogen atom has been replaced with at least one non-hydrogen group, such as a hydrocarbyl group, a heteroatom, or a heteroatom containing group, or at least one functional group such as a halogen (e.g., Br, Cl, F, I), —NR*₂, —NR*—CO—R*, —OR*, *—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

The term “substituted hydrocarbyl” means a hydrocarbyl radical in which at least one hydrogen atom of the hydrocarbyl radical has been substituted with at least one heteroatom (such as halogen, e.g., Br, Cl, F or I) or heteroatom-containing group (such as a functional group, e.g., —NR*₂, —NR*—CO—R*, —OR*, *—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

Other examples of functional groups include those typically referred to as amines, imides, amides, ethers, alcohols (hydroxides), sulfides, sulfates, phosphides, halides, phosphonates, alkoxides, esters, carboxylates, aldehydes, and the like.

Unless otherwise indicated, room temperature is 23° C. “Different” or “not the same” as used to refer to R groups in any formula herein (e.g., R² and R⁸ or R⁴ and R¹⁰) or any substituent herein indicates that the groups or substituents differ from each other by at least one atom or are different isomerically.

Unless otherwise noted, all molecular weights are reported in units of g/mol or Daltons (Da). The following abbreviations may be used herein: Me is methyl, Et is ethyl, Pr is propyl, cPr is cyclopropyl, nPr is n-propyl, iPr is isopropyl, Bu is butyl, nBu is normal butyl, iBu is isobutyl, sBu is sec-butyl, tBu is tert-butyl, Oct is octyl, Ph is phenyl, Bn is benzyl, CBD refers to cannabidiol, THC refers to tetrahydrocannabinol, TPGS refers to tocopheryl polyalkylene glycol succinates and derivatives thereof, HPMC refers to hydroxypropyl methylcellulose and derivatives thereof, and the like.

For purposes herein, the terms “group,” “radical,” and “substituent” may be used interchangeably. A multivalent radical refers to a radical having two or more attachment points, e.g., methylene —CH₂— is a multivalent radical of methane.

Unless indicated otherwise, as used herein, a water soluble composition is defined as a composition in which 400 mg of the composition dissolves, (i.e., forms a clear solution) in 240 ml of water at a temperature of 20° C. with stirring within 30 seconds.

Unless indicated otherwise, as used herein, a water miscible composition is defined as a composition in which 400 mg of the composition disperses (i.e., forms a clear to turbid solution) in 240 ml of water at a temperature of 20° C. with stirring within 30 seconds, and in which at least 95 wt % of the composition remains dispersed in the mixture after 5 minutes without stirring.

As used herein, “colloid” refers to a mixture containing two phases, a dispersed phase and a continuous phase, with the dispersed phase containing particles (droplets) distributed throughout the continuous phase. Colloidal mixtures include aerosols, foams, and dispersions, for example, emulsions, for example, nanoemulsions. A liquid colloid, for example, a nanoemulsion, can have a similar appearance, for example, similar clarity, to a solution in which there is no dispersed phase.

As used herein, “emulsion” refers to a colloidal dispersion of two immiscible liquids, for example, an oil and water (or other aqueous liquid, e.g., a polar solvent), one of which is part of a continuous phase and the other of which is part of a dispersed phase. Dilution of the instant composition results in an emulsion, preferably an oil-in-water nanoemulsions in which the oil phase, (i.e., the cannabinoids) is the dispersed phase and the polar water phase is the continuous phase. Emulsions typically are stabilized by one or more surfactants and/or co-surfactants and/or emulsion stabilizers. Surfactants form an interfacial film between the oil and water phase of the emulsion, providing stability. In some embodiments, the nanoemulsions formed by dilution of the instant composition in water include micelles that contain one or more surfactants surrounding the cannabinoids and/or other non-polar ingredient which are dispersed in the water phase.

As used herein, a “nanoemulsion” is an emulsion in which the dispersed droplets have particle size of less than 1000 nm or less than about 1000 nm, typically, less than 500 nm or less than about 500 nm, typically less than 300 nm or less than about 300 nm, for example, less than 250 nm or less than about 250 nm, for example, less than or less than about 200 nm, for example, less than or less than about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm. Exemplary of nanoemulsions are those formed when embodiments of the composition are diluted in water. Likewise, the particles of the composition have an average particle size of less than 1000 nm or less than about 1000 nm, typically, less than 500 nm or less than about 500 nm, typically less than 300 nm or less than about 300 nm, for example, less than 250 nm or less than about 250 nm, for example, less than or less than about 200 nm, for example, less than or less than about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm.

As used herein, discrete particles refer to individual domains comprising the cannabinoids and the carrier. They may be in the form of a powder and/or dispersed on a substrate. The particle size refers to the average particle size, which may be calculated by various methods readily known in the art. Suitable methods for determining the average particle size include examination of either an SEM or AFM micrograph or image in which the number average particle size may be determined. For purposes herein, the size of any one particle is always determined along the longest axis of the particle. Accordingly, for purposes herein, the terms “particle size”, “average particle size”, “average maximum dimension” and the like are used interchangeably. Particle size diameter can be expressed in terms of a unit of length, for example, nanometers (nm). Alternatively, information about particles in embodiments of the particulate composition or the mixture produced by dilution thereof can be expressed in terms of particle density, for example, ppm (parts per million), or percent solids, in the compositions.

As used herein, “surfactant” refers to synthetic and naturally occurring amphiphilic molecules that have hydrophobic portion(s) and hydrophilic portion(s). A “surfactant system” refers to combinations and/or blends or mixtures of surfactants to produce an intended characteristic. Examples of surfactant systems include so-called “matched pairs” of surfactants having different hydrophobe/lipophobe balance (HLB) characteristics.

As used herein, “HLB” refers to a value that is used to index and describe a surfactant according to its relative hydrophobicity/hydrophilicity, relative to other surfactants. A surfactant's HLB value is an indication of the molecular balance of the hydrophobic and hydrophilic portions of the surfactant, which is an amphipathic molecule. Each surfactant and mixture of surfactants (and/or co-surfactants) has an HLB value that is a numerical representation of the relative weight percent of hydrophobic and hydrophilic portions of the surfactant molecule(s). HLB values are derived from a semi-empirical formula. The relative weight percentages of the hydrophobic and hydrophilic groups are indicative of surfactant properties, including the molecular structure, for example, the types of aggregates the surfactants form and the solubility of the surfactant. See, for example, Griffin (1949) J. Soc. Cos. Chem. 1:311. Surfactant HLB values range from 1-45, while the range for non-ionic surfactants typically is from 1-20. The more lipophilic a surfactant is, the lower its HLB value. Conversely, the more hydrophilic a surfactant is, the higher its HLB value.

Due to their amphiphilic (amphipathic) nature, surfactants typically can reduce the surface tension between two immiscible liquids, for example, the oil and water phases in an emulsion, stabilizing the emulsion. Surfactants may be characterized herein based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10.

As used herein, “co-surfactant” is used to refer to a surfactant that is used in the provided compositions in combination with the primary surfactant, for example, the particulate composition described herein, for example, to improve the emulsification of the provided compositions and/or compounds, for example, to emulsify the ingredients upon dilution. In one example, the provided compositions can contain at least one surfactant and at least one co-surfactant. Typically, the co-surfactant represents a lower percent, by weight of the provided compositions, compared to the surfactant. Thus, the provided compositions typically have a lower concentration of the co-surfactant(s) than of the surfactant.

As used herein, “micelle” refers to aggregates formed by surfactants that typically form when a surfactant is present in an aqueous composition, typically when the surfactant is used at a concentration above the critical micelle concentration (CMC). In micelles, the hydrophilic portions of the surfactant molecules contact the aqueous or the water phase, while the hydrophobic portions form the core of the micelle, which can encapsulate the non-polar cannabinoids and other ingredient(s). Typically, the surfactants form micelles containing the cannabinoids within either as the particles are formed, upon dilution of embodiments of the particulate composition or the mixture produced by dilution thereof in water, or both. Typically, the micelles in embodiments of the particulate composition or the mixture produced by dilution thereof have an average particle size of less than or equal to about 1000 nm, typically less than or less than about 500 nm, typically less than 300 or less than about 300 nm, for example, less than 250 nm or less than about 250 nm, for example, less than 200 nm or less than about 200 nm, for example, less than or less than about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm.

As used herein, “analog” and/or “derivative” refers to a chemical compound that is structurally similar to another compound (referred to as a parent compound), but differs slightly in composition, for example, due to the variation, addition or removal of an atom, one or more units (e.g., methylene units, —(CH₂)ₙ—) or one or more functional groups, e.g., a glycoside of a phenolic compound is a phenolic ether analog and/or derivative of the phenolic parent compound. The analog and/or derivative can have different chemical or physical properties compared with the original compound and/or can have improved biological and/or chemical activity. Alternatively, the analog and/or derivative can have similar or identical chemical or physical properties compared with the original compound and/or can have similar or identical biological and/or chemical activity. For example, the analog and/or derivative can be more hydrophilic, or it can have altered reactivity as compared to the parent compound. The analog and/or derivative can mimic the chemical and/or biological activity of the parent compound (i.e., it can have similar or identical activity), or, in some cases, can have increased or decreased activity. The analog and/or derivative can be a naturally or non-naturally occurring (e.g., synthetic) variant of the original compound. Other types of analogs and/or derivatives include isomers (e.g., enantiomers, diastereomers) and other types of chiral variants of a compound, as well as structural isomers. The analog and/or derivative can be a branched or cyclic variant of a linear compound. For example, a linear compound can have an analog and/or derivative that is branched or otherwise substituted, e.g., a saccharide, to impart certain advantageous properties (e.g., improved hydrophobicity or bioavailability).

As used herein, “organoleptic properties” refers to sensory attributes of a food or beverage, in particular upon dilution of the particulate composition into a beverage. Those of skill in the art understand such properties and they can be quantitated if needed. Organoleptic properties include, but are not limited to, taste, odor and/or appearance. “Desirable” or “advantageous” organoleptic properties include those organoleptic properties of a food or beverage composition for consumption by an average human subject, such as a desirable odor, taste and/or appearance, or the lack of an undesirable odor, taste and/or appearance. Undesirable organoleptic properties include the presence of, for example, an undesirable taste, odor or appearance attribute, such as the presence of an “off-taste” or “off-odor,” for example a fishy, grassy, metal or iron, sharp or tingling taste or odor, or the presence of an undesirable appearance attribute, such as separation or precipitation. In one example, the provided beverage compositions retain the same or about the same taste, odor and/or appearance as the same beverage composition that does not contain the composition according to embodiments disclosed herein. As such, dilution of a composition according to one or more embodiments disclosed herein results in a beverage or other consumable material which retains organoleptic properties desirable for consumption by an average human subject. Desirable and undesirable organoleptic properties can be measured by a variety of methods known to those skilled in the art, including, for example, organoleptic evaluation methods by which undesirable properties are detectable by sight, taste and/or smell and chemical tests, as well as by chemical analytical methods. As used herein, “retaining the organoleptic properties” refers to retention of these properties upon storage for a recited period of time, typically at room temperature.

As used herein, “visible particles” are particles, for example, in a liquid, such as an emulsion, that are visible when viewing the liquid with the naked eye (i.e., without magnification). For example, the visible particles can be particles that are observed by the artisan formulating embodiments of the particulate composition or the mixture produced by dilution thereof. In one example, the dilution of the particulate composition contain no visible particles. In another example, the diluted compositions contain few visible particles, for example, no more visible particles than another liquid, for example, a beverage. The presence of visible particles and the number of visible particles is determined by empirical observation.

As used herein, “clear” can be used to describe the resultant mixture upon dilution of the compositions provided herein, for example, dilution of the particulate composition in an aqueous liquid produces a nanoemulsion which is a clear liquid, i.e., one that does not appear cloudy by empirical observation, such as to the naked eye, and/or does not contain particles or crystals that are visible to the naked eye, or that does not exhibit “ringing.” For example, a liquid can be described as clear when the dispersed particles have an average particle size of less than or about 200 nm.

As used herein, “stability” refers to a desirable property of the provided particulate composition and/or the liquid dilution of the particulate composition. For example, the ability of the provided particulate composition or the liquid dilution of the particulate composition to remain free from one or more changes over a period of time, for example, at least or longer than 1 day, 1 week, 1 month, 1 year, or more. For example, a particulate composition can be described as stable if it is formulated such that it remains free from oxidation or substantial oxidation over time, and/or desirable for human consumption over time, has a lack of precipitates forming over time, does not exhibit any visible phase separation over a period of time.

As used herein, “stabilize” means to increase the stability of one of the provided compositions.

As used herein, “hydrophilic” and “polar” refer synonymously to ingredients and/or compounds having greater solubility in aqueous liquids, for example, water, than in fats, oils and/or organic solvents (e.g., methanol, ethanol, ethyl ether, acetone and benzene).

As used herein, a “solvent” is an ingredient that can be used to dissolve another ingredient. Solvents include polar and non-polar solvents. Non-polar solvents include oils and other non-polar ingredients that dissolve non-polar compounds. Typically, the non-polar solvent included in embodiments of the particulate composition or the mixture produced by dilution thereof is an oil. The non-polar solvent typically is not the non-polar ingredient itself, i.e., is distinct from the cannabinoid. More than one non-polar solvent can be used. Certain compounds, for example, flaxseed oil and safflower oil, can be non-polar solvents and non-polar ingredients. Typically, the non-polar solvent contains one or more oils, typically oils other than the non-polar ingredient or oil(s) not contained in the non-polar ingredient. Exemplary non-polar solvents include, but are not limited to, oils (in addition to the non-polar ingredient), for example, tocopheryl polyalkylene glycol oil, flaxseed oil, CLA, borage oil, rice bran oil, D-limonene, canola oil, corn oil, MCT oil and oat oil. Other oils also can be used.

As used herein, “polar solvent” refers to a solvent that is readily miscible with water and other polar solvents. Polar solvents are well-known and can be assessed by measuring any parameter known to those of skill in the art, including dielectric constant, polarity index and dipole moment (see, e.g., Przybitek (1980) “High Purity Solvent Guide,” Burdick and Jackson Laboratories, Inc.). For example, polar solvents generally have high dielectric constants, such as greater than or about 15, generally have high polarity indices, typically greater than or about 3, and generally large dipole moments, for example, greater than or about 1.4 Debye. Polar solvents include polar protic solvents and polar aprotic solvents.

As used herein, a “polar protic solvent” is a polar solvent containing a hydrogen atom attached to an electronegative atom, such that the hydrogen has a proton-like character and/or the bond between the hydrogen and electronegative atom is polarized. Exemplary polar protic solvents include, but are not limited to, water, alcohols, including monohydric, dihydric and trihydric alcohols, including, but not limited to, methanol, ethanol, glycerin and propylene glycol.

As used herein, “monohydric alcohols” are alcohols that contain a single hydroxyl group including, but not limited to, methanol, ethanol, propanol, isopropanol, n-butanol and t-butanol.

As used herein, “dihydric alcohols” are alcohols that contain two hydroxyl groups. Exemplary dihydric alcohols include, but are not limited to, glycols, e.g., propylene glycol, ethylene glycol, tetraethylene glycol, triethylene glycol and trimethylene glycol.

As used herein, “trihydric alcohols” are alcohols that contain three hydroxyl groups. Exemplary trihydric alcohols include, but are not limited to, glycerin, butane-1,2,3-triol, pentane-1,3,5-triol and 2-amino-2-hydroxymethyl-propane-1,3-diol.

As used herein, “non-polar,” “lipophilic” and “lipid-soluble” synonymously refer to compounds and/or ingredients, for example, non-polar compounds and non-polar ingredients, which have greater solubility in organic solvents (e.g., ethanol, methanol, ethyl ether, acetone and benzene), fats and oils than in aqueous liquids, for example, water. Non-polar ingredients include drugs, hormones, vitamins, nutrients and other lipophilic compounds. Typically, non-polar ingredients are poorly water-soluble, for example, water insoluble or compounds having low water solubility. Exemplary non-polar ingredients include ingredients that contain one or more non-polar compounds, for example, lipid-soluble drugs, hormones, essential fatty acids, for example, polyunsaturated fatty acids (PUFA), for example, omega-3 and omega-6 fatty acids, vitamins, nutrients, nutraceuticals, minerals and other compounds. Additional exemplary non-polar ingredients are described herein. The provided compositions can be formulated with any non-polar ingredient, for example, any non-polar ingredient that is or contains a non-polar compound.

As used herein, an “additive” includes anything other than cannabinoids that one can add to a food, beverage, or other human consumable to enhance one or more of its nutritional, pharmaceutical, dietary, health, nutraceutical, health benefit, energy-providing, treating, holistic, or other properties. For example, the additives can be oil-based additives (e.g., non-polar ingredients), such as nutraceuticals; pharmaceuticals; vitamins, for example, oil-soluble vitamins, e.g., vitamin D, tocopheryl polyalkylene glycol and vitamin A; minerals; fatty acids, such as essential fatty acids, for example, polyunsaturated fatty acids, e.g., omega-3 fatty acids and omega-6 fatty acids, such as alpha-linolenic acid (ALA), docosahexaenoic acid (DHA), eicosapentaenoic acid (EPA), gamma-linolenic acid (GLA), CLA, saw palmetto extract, flaxseed oil, fish oil and algae oil. phytosterols; coenzymes, such as coenzyme Q10; and any other oil-based additives.

As used herein, “water insoluble” refers to a compound that does not dissolve when the compound is mixed with water, for example, when mixed with water at room temperature, for example, between or between about 25° C. and 50° C.

As used herein, “low water solubility” refers to a compound that has a solubility in water of less than or about 30 mg/mL, for example, when mixed with water at room temperature, such as between or between about 25° C. and 50° C. As used herein, “poorly water-soluble” can be used to refer to compounds, for example, non-polar compounds, that are water insoluble or have low water solubility.

As used herein, “food and beverage product” refers to a product that is suitable for human consumption. For example, “food and beverage product” can refer to a composition that is dissolved in a solvent, typically an aqueous solvent, e.g., water, to form a liquid dilution composition, i.e., beverage composition or beverage product. “Food and beverage product” can also refer to the final product that is suitable for human consumption, such as the liquid dilution composition, i.e., beverage composition or beverage product.

As used herein, a “beverage base” refers to an aqueous composition to which one or more non-polar ingredients can be added. A beverage base includes, but is not limited to, an aqueous composition that contains one or more of a polar solvent, typically water, a juice, such as a fruit juice, a fruit juice concentrate, a fruit juice extract, a fruit flavor, a soda, a flavored soda, a carbonated water, a carbonated juice and any combination thereof. Embodiments of the particulate composition can be introduced into a beverage base (or beverage or other food).

As used herein, a “fruit juice,” “fruit juice concentrate,” “fruit juice extract” or “fruit flavor” refer to fruit-based juices and flavors that impart taste or smell to the provided beverage compositions (products). Any juice or fruit flavor can be added to the provided beverage compositions, including, but not limited to, plum, prune, date, currant, fig, grape, raisin, cranberry, pineapple, peach, nectarine, banana, apple, pear, guava, apricot, Saskatoon berry, blueberry, plains berry, prairie berry, mulberry, elderberry, Barbados cherry (acerola cherry), choke cherry, chocolate, vanilla, caramel, coconut, olive, raspberry, strawberry, huckleberry, loganberry, dewberry, boysenberry, kiwi, cherry, blackberry, honey dew, green tea, cucumber, quince, buckthorn, passion fruit, sloe, rowan, gooseberry, pomegranate, persimmon, mango, rhubarb, papaya, litchi, lemon, orange, lime, tangerine, mandarin and grapefruit juices, or any combination thereof. Exemplary beverage compositions provided herein include combinations of juices or flavors that impart peach mango, peach, citrus, pomegranate blueberry, tropical berry, cherry chocolate, vanilla, cherry vanilla, chocolate blueberry, chocolate caramel, cucumber, green tea, honey-dew melon, pineapple papaya, peach nectarine, raspberry lemonade, grape, orange tangerine, orange, lime and mixed berry flavors.

As used herein, “fatty acid” refers to straight-chain hydrocarbon molecules with a carboxyl (—COOH) group at one end of the chain.

As used herein, “polyunsaturated fatty acid” and “PUFA” are used synonymously to refer to fatty acids that contain more than one carbon-carbon double bonds in the carbon chain of the fatty acid. PUFAs, particularly essential fatty acids, are useful as dietary supplements.

Examples include omega-3 fatty acids such as alpha-linolenic acid (alpha-linolenic acid; ALA) (18:3omega3) (a short-chain fatty acid); stearidonic acid (18:4omega3) (a short-chain fatty acid); eicosapentaenoic acid (EPA) (20:5omega3); docosahexaenoic acid (DHA) (22:6omega3); eicosatetraenoic acid (24:4omega3); docosapentaenoic acid (DPA, clupanodonic acid) (22:5omega3); 16:3 omega3; 24:5 omega3 and nisinic acid (24:6omega3). Longer chain omega-3 fatty acids can be synthesized from ALA (the short-chain omega-3 fatty acid). Exemplary of non-polar ingredients containing omega-3 fatty acids are non-polar ingredients containing DHA and/or EPA, for example, containing fish oil, krill oil and/or algae oil, for example, microalgae oil, and non-polar ingredients containing alpha-linolenic acid (ALA), for example, containing flaxseed oil. Other exemplary fatty acids include linoleic acid (18:2omega6) (a short-chain fatty acid); gamma-linolenic acid (GLA) (18:3omega6); dihomo gamma linolenic acid (DGLA) (20:3omega6); eicosadienoic acid (20:2omega6); arachidonic acid (AA) (20:4omega6); docosadienoic acid (22:2omega6); adrenic acid (22:4omega6); and docosapentaenoic acid (22:5omega6). Exemplary of non-polar ingredients containing omega-6 fatty acids are ingredients containing GLA, for example, borage oil. Also exemplary of omega-6-containing non-polar ingredients are compounds containing conjugated fatty acids, for example, conjugated linoleic acid (CLA) and compounds containing saw palmetto extract.

As used herein, “preservative” and “preservativer” are used synonymously to refer to ingredients that can improve the stability of embodiments of the particulate composition and/or the liquid produced by dilution of the particulate composition. Preservatives, particularly food and beverage preservatives, are well known. Any known preservative can be used in embodiments of the particulate composition and/or the liquid produced by dilution of the particulate composition. Exemplary of the preservatives include benzyl alcohol, benzyl benzoate, methyl paraben, propyl paraben, antioxidants, for example, vitamin E, vitamin A palmitate and beta carotene. Typically, a preservative is selected that is safe for human consumption, for example, in foods and beverages, for example, a GRAS certified and/or Kosher-certified preservative, for example, benzyl alcohol.

As used herein, an “antioxidant” refers to a stabilizer or one component of a stabilizing system that acts as an antioxidant, and that, when embodiments of the particulate composition are added to a beverage composition in combination with the other required components (i.e., acid and/or bicarbonate or carbonate) yields beverage compositions that retain one or more desired organoleptic properties, such as, but not limited to, the taste, smell, odor and/or appearance, of the beverage composition over time. Typically, antioxidants are food-approved, e.g., edible antioxidants, for example, antioxidants that are safe and/or approved for human consumption. Exemplary antioxidants include, but are not limited to, ascorbic acid, vitamin C, ascorbate and coenzyme Q-containing compounds, including, but not limited to, coenzyme Q10.

As used herein, an “acid” or “ingestible acid” refers to a stabilizer or one component of a stabilizing system that, when added to a beverage composition in combination with the other components (i.e., antioxidant and/or bicarbonate or carbonate), yields compositions that retain one or more desired organoleptic properties, such as, but not limited to, the taste, smell, odor and/or appearance of the composition over time. Typically, the acids are food-approved, e.g., edible acids or ingestible acids, for example, acids that are safe and/or approved for human consumption. Exemplary acids include, but are not limited to, citric acid, phosphoric acid, adipic acid, ascorbic acid, lactic acid, malic acid, fumaric acid, gluconic acid, succinic acid, tartaric acid and maleic acid.

As used herein, a “bicarbonate” or “carbonate” refers to a stabilizer or one component of a stabilizing system that, when added to a beverage composition in combination with the other components (i.e., the acid and/or antioxidant) yields compositions that retain one or more desired organoleptic properties, such as, but not limited to, the taste, smell, odor and/or appearance of the composition over time. Typically, bicarbonates or carbonates are food-approved, e.g., edible bicarbonates or carbonates, for example, bicarbonates or carbonates that are safe and/or approved for human consumption. Exemplary bicarbonates include, but are not limited to, potassium bicarbonate and sodium bicarbonate. Exemplary carbonates include, but are not limited to, potassium carbonate, sodium carbonate, calcium carbonate, magnesium carbonate and zinc carbonate.

As used herein, “emulsion stabilizer” refers to compounds that can be used to stabilize and/or emulsify and/or change the viscosity of embodiments of the particulate composition and/or the liquid produced by dilution of the particulate composition. For example, the emulsion stabilizer can increase the viscosity of the liquid produced by dilution of the particulate composition. One or more emulsion stabilizers can be used. Addition of an emulsion stabilizer can prevent separation of the particulate composition and/or the liquid produced by dilution of the particulate composition.

As used herein, a “pH adjuster” is any compound, typically an acid or a base, that is capable of changing the pH of embodiments of the particulate composition or the mixture produced by dilution thereof, for example, to reduce the pH of the particulate composition and/or the liquid produced by dilution of the particulate composition, or to increase the pH of the same, typically without altering other properties of the particulate composition and/or the liquid produced by dilution of the particulate composition, or without substantially altering other properties. pH adjusters are well known. Exemplary of the pH adjusters are acids, for example, citric acid and phosphoric acid, and bases.

As used herein, “flavor” is any ingredient that changes, typically improves, the taste and/or smell of embodiments of the particulate composition and/or the liquid produced by dilution of the particulate composition, for example, in a beverage.

As used herein, “natural” is used to refer to a composition, and/or ingredients in embodiments of the particulate composition and/or the liquid produced by dilution of the particulate composition, that can be found in nature and is not solely man-made. For example, benzyl alcohol is a natural preservative. Similarly, tocopheryl polyethylene glycol is a natural surfactant. The natural composition/ingredient can be GRAS and/or Kosher-certified.

As used herein, “G.R.A.S.” and “GRAS” are used synonymously to refer to compounds, compositions and ingredients that are “Generally Regarded as Safe” by the USDA and FDA for use as additives, for example, in foods, beverages and/or other substance for human consumption, such as any substance that meets the criteria of sections 201(s) and 409 of the U.S. Federal Food, Drug and Cosmetic Act. Typically, embodiments of the particulate composition and/or the liquid produced by dilution of the particulate composition disclosed herein are GRAS certified. Likewise, “kosher” is used to refer to substances that conform to Jewish Kosher dietary laws, for example, substances that do not contain ingredients derived from non-kosher animals or do not contain ingredients that were not made following kosher procedures. Typically, embodiments of the particulate composition and/or the liquid produced by dilution of the particulate composition are Kosher-certified.

As used herein, “excipients”, refer to any substance needed to formulate the particulate composition to a desired form. For example, suitable excipients include but are not limited to, diluents or fillers, binders or granulating agents or adhesives, disintegrates, lubricants, antiadherants, glidants, wetting agents, dissolution retardants or enhancers, adsorbents, buffers, chelating agents, preservatives, colors, flavors and sweeteners. Typical excipients include, but are not limited to, starch, pregelatinized starch, maltodextrin, monohydrous dextrose, alginic acid, sorbitol and mannitol. In general, the excipient should be selected from non-toxic excipients (IIG, Inactive Ingredient Guide, or GRAS, Generally Regarded as safe, Handbook of Pharmaceutical Excipients).

As used herein, a binder is an excipient added to a composition to aid formation of a powder when the particulate composition is dried. Non-limiting examples of suitable binders include, but are not limited to, acacia, dextrin, starch, povidone, carboxymethylcellulose, guar gum, glucose, hydroxypropyl methylcellulose, methylcellulose, polymethacrylates, maltodextrin, hydroxyethyl cellulose, whey, disaccharides, sucrose, lactose, polysaccharides and their derivatives such as starches, cellulose or modified cellulose such as microcrystalline cellulose and cellulose ethers such as hydroxypropyl cellulose, sugar alcohols such as xylitol, sorbitol or maltitol, protein, gelatins and synthetic polymers, such as polyvinylpyrrolidone (PVP) or polyethylene glycol (PEG).

As used herein, “homolog” refers to an analog that differs from the parent compound only by the presence or absence of a simple unit, such as a methylene unit, or some multiple of such units, e.g., —(CH₂)ₙ—. Typically, a homolog has similar chemical and physical properties as the parent compound. Exemplary of the homologs used in the provided compositions and methods are TPGS homologs.

As used herein, pharmaceutical compositions comprising embodiments of the composition refer to compositions formulated for administration in a pharmaceutical carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this disclosure as a unit-dose formulation, for example, a tablet, which may contain from about 0.01 or 0.5% to about 95% or 99% by weight of the cannabinoid(s) component. Furthermore, a “pharmaceutically acceptable” component such as a salt, carrier, excipient or diluent of a composition according to the instant disclosure is a component that (i) is compatible with the other ingredients of the composition in that it can be combined with the compositions of the present disclosure without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

As used herein, a biocompatible, biodegradable polymer is a polymer which can be broken down in vivo to monomer and/or oligomer fragments, wherein the monomeric or oligomeric fragments do not provoke an immune response, are not toxic, and can be easily excreted.

As used herein, a nanofiber refers to a fiber having a length along the longest dimension, and a width perpendicular to the length, wherein a maximum average width along the entire length of the fiber is less than 1 micron.

### Compositions

In embodiments, a composition comprising a plurality of discrete particles comprising one or more cannabinoids disposed at least partially within a polymeric carrier having a maximum overall dimension of less than 1 micron.

In one or more embodiments, a composition comprising a plurality of discrete nanofibers comprising one or more cannabinoids disposed at least partially within a polymeric carrier.

In one or more embodiments, a process to produce a composition comprising the steps of: a) providing one or more precursor mixtures comprising one or more cannabinoids in a solvent and one or more polymeric carrier components, preferably in a solvent; b) electrospraying these one or more precursor mixtures under electrospray conditions to form a plurality of discrete particles comprising one or more cannabinoids at least partially encapsulated with or disposed on the polymeric carrier, wherein each of said discrete particles having a maximum dimension of less than or equal to about 1 micron, preferably less than or equal to about 0.5 microns, preferably less than or equal to about 100 nanometers, preferably less than or equal to about 50 nm, and/or agglomerates of said discrete particles.

In one or more embodiments, the first precursor mixture comprises one or more cannabinoids in a solvent; the second precursor mixture comprises one or more polymeric carrier components dissolved and/or dispersed in a solvent; and each of the precursor mixtures are coaxially electrosprayed under electrospray conditions to form a plurality of discrete particles comprising one or more cannabinoids at least partially encapsulated within the polymeric carrier.

In one or more embodiments, a process to produce a composition comprising the steps of: a) providing one or more precursor mixtures comprising one or more cannabinoids in a solvent and one or more polymeric carrier components, preferably in a solvent; and b) electrospinning these one or more precursor mixtures under electrospinning conditions to form a plurality of nanofibers comprising one or more cannabinoids at least partially encapsulated with or disposed on the polymeric carrier.

In one or more embodiments of the process, the first precursor mixture comprises one or more cannabinoids in a solvent; the second precursor mixture comprises one or more polymeric carrier components dissolved and/or dispersed in a solvent; and each of the precursor mixtures are coaxially electrospun under electrospinning conditions to form a plurality of nanofibers comprising one or more cannabinoids at least partially encapsulated within the polymeric carrier.

In an embodiment, a composition comprises a plurality of discrete particles comprising one or more cannabinoids disposed at least partially within a polymeric carrier having a maximum overall dimension of less than 1 micron. In some embodiments, the composition is produced by electrospray of a solution comprising one or more cannabinoids and the polymeric carrier. In some embodiments, the composition is produced by coaxial electrospray including an outer flow comprising the polymeric carrier, and an inner flow comprising the one or more cannabinoids.

In embodiments, the composition includes one or more cannabinoids include a tetrahydrocannabinol (THC), a cannabidiol (CBD), a cannabivarin (CBV), a tetrahydrocannabivarin (THCV), or a combination thereof.

In some embodiments of the composition, the polymeric carrier includes a gelatin, ethyl cellulose, or a combination thereof.

In embodiments the composition comprises greater than or equal to about 30 wt % of the one or more cannabinoids, or greater than or equal to about 40 wt %, or greater than or equal to about 50 wt %, or greater than or equal to about 60 wt %, or greater than or equal to about 70 wt % of the one or more cannabinoids.

In embodiments, a 10 wt % mixture of the composition in water at 25° C. forms a clear solution.

In other embodiments, a composition comprises a plurality of discrete nanofibers comprising one or more cannabinoids disposed at least partially within a polymeric carrier having a maximum width of less than 1 micron. In some embodiments, the composition is produced by electrospinning of a solution comprising one or more cannabinoids and the polymeric carrier. In some embodiments, the composition is produced by coaxial electrospinning including an outer flow comprising the polymeric carrier, and an inner flow comprising the one or more cannabinoids.

In such embodiments of the composition, the one or more cannabinoids include a tetrahydrocannabinol (THC), a cannabidiol (CBD), a cannabivarin (CBV), a tetrahydrocannabivarin (THCV), or a combination thereof. In some embodiments, the polymeric carrier includes a gelatin, ethyl cellulose, or a combination thereof. In some of such embodiments, the composition comprises greater than or equal to about 30 wt % of the one or more cannabinoids, or greater than or equal to about 40 wt %, or greater than or equal to about 50 wt %, or greater than or equal to about 60 wt %, or greater than or equal to about 70 wt % of the one or more cannabinoids.

In some of such embodiments, a 10 wt % mixture of the composition in water at 25° C. forms a clear solution.

In one or more embodiments, a process to produce a composition comprising the steps of providing one or more precursor mixtures comprising one or more cannabinoids and one or more polymeric carrier components in a solvent and electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles. In some embodiments, a process to produce a composition comprising the steps of providing one or more precursor mixtures comprising one or more cannabinoids and one or more polymeric carrier components in a solvent and electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.

In some embodiments of the process, a first precursor mixture comprises one or more cannabinoids in a solvent and a second precursor mixture comprises one or more polymeric carrier components dissolved and/or dispersed in a solvent.

### Electro Spraying and Electro Spinning

In one or more embodiments according to the instant disclosure, the composition is formed by electrospraying and/or electrospinning at least one precursor composition comprising one or more cannabinoids, a polymeric carrier, and a solvent. Preferably at least a portion of the polymeric carrier is water soluble and/or water miscible.

In one or more embodiments of the disclosure, a process comprises the steps of combining one or more cannabinoids and/or derivatives thereof, and one or more water soluble and/or water miscible carrier in a solvent to form a first precursor composition; electrospraying the first composition to form a plurality of discrete particles comprising one or more cannabinoids and/or derivatives thereof disposed within or on the carrier, each of said discrete particles having a maximum dimension of less than or equal to about 1 micron, preferably less than or equal to about 0.5 microns, preferably less than or equal to about 100 nm, preferably less than or equal to about 50 nm, preferably less than or equal to about 10 nm, and/or agglomerates comprising a plurality of the discrete particles.

In one or more embodiments of the disclosure, a process comprises the steps of combining one or more cannabinoids and/or derivatives thereof, and one or more water soluble and/or water miscible carrier in a solvent to form a first precursor composition; electrospinning the first precursor composition to form a plurality of nanofibers comprising one or more cannabinoids at least partially disposed within and/or on a polymeric carrier produced via electrospinning deposition of a precursor mixture. Preferably, the polymeric carriers are water soluble and/or water miscible.

Electrostatic atomization, also referred to as electrospray, refers to the atomization of a liquid through the Coulombic interaction of charges and the applied electric field. Applicant has discovered that electrostatic atomization offers several advantages over alternative atomization techniques. This is mainly due to the net charge on the surface of the droplets that is generated and the Coulombic repulsion of the droplets. This net charge causes the droplets to self-disperse, preventing their coalescence. The trajectory of a charged droplet can be guided by an electrostatic field. The advantage of this type of atomization is the ability to control the size distribution of the spray and under specific operating conditions, obtain a monodisperse spray. Because of these advantages, there are a wide number of applications where electrostatic atomization can be used.

Electrospray can be described by three different processes. The first process is the formation of the liquid meniscus at a capillary tip which results from a number of forces acting on the interface, including surface tension, gravitational, electrostatics, inertial, and viscous forces. Sir Geoffrey Taylor was the first to calculate analytically a conical shape for the meniscus through the balance of surface tension and electrical normal stress forces which we now know is called the “Taylor cone” in electrospray and appears in the cone jet mode.

The cone jet mode is one of the most widely studied and used modes of electrospray. In the cone-jet mode liquid leaves the capillary in the form of an axi-symmetric cone with a thin jet emitted from its apex. The small jet of liquid issuing out of the nozzle is electrostatically charged when subjected to an intense electric field at the tip of the capillary nozzle. In this case, the droplets are approximately 10 microns in diameter and difficult to visualize with standard macro photography. The charged droplets are propelled away from the nozzle by the Coulomb force and are dispersed out as a result of charge on the droplets.

As used herein, the term “Taylor cone” refers to the phenomenon wherein when a small volume of liquid is exposed to an electric field such that the shape of the liquid starts to deform from the shape caused by surface tension alone. As the voltage is increased the effect of the electric field becomes more prominent. As the electric field approaches exerting a similar amount of force on the droplet as the surface tension does, a cone shape begins to form with convex sides converging to a pointed tip. When a certain threshold voltage has been reached the slightly pointed tip inverts and emits a jet of liquid. This is called a cone-jet and is the beginning of the electrospraying process resulting in the formation of the particulates according to embodiments disclosed herein.

Accordingly, the embodiments of the composition disclosed herein are produced via electrospraying and electrospinning, which refers to methods of forming discrete particles and fibers, respectively, which utilizes the ability of an electric field to overcome the surface tension of a solvent, polymer or biomacromolecule solution (or melt). In the electrospray process utilized herein, an electric potential is selected and applied to an electrospray nozzle through which the precursor mixture flows, to form charged droplets which are generally collected on a collection plate. A typical electrospray system includes a pump connected to hollow capillary tube. A high voltage power supply i.e., 1 kV or higher, is connected to the hollow capillary tube, a portion of which is constructed from metal. Electric potential supplied to the hollow capillary tube in turn imparts a charge to a liquid passing therethrough. As the liquid is pumped through the hollow capillary tube and exits through a nozzle located at the end of the tube, columbic interactions cause the liquid to break apart into charged droplets. These charged droplets are then collected as particles on collection target which is at a lower potential than that of the capillary, typically the collector is at ground.

In embodiments, electrospraying includes feeding the liquid comprising the cannabinoid through a hollow capillary tube terminated by a nozzle, which for brevity herein is simply referred to as a nozzle, into an external medium onto a grounded electrode which serves as the collector. The external medium may be a gas, e.g., air, at a pressure which may be atmospheric pressure, above atmospheric pressure, or in the alternative the liquid may be electrosprayed into a partial vacuum. In alternative embodiments, the liquid is electrosprayed into a liquid, typically a dielectric liquid. Depending on various process parameters, such as flow rate and the electric voltage applied between the needle and a grounded electrode, the liquid meniscus at the end of the needle adopts a conical shape resulting from the balance between the capillary and the electrohydrodynamic normal stresses. This conical shape is referred to as a Taylor cone. Eventually, a micro- or nanometric jet issues from the tip of the Taylor cone, which will eventually break up forming a spray (or hydrosol) of charged droplets. The droplets are collected on the collection target as particles.

The external environment may be at ambient temperature or may be heated to facilitate evaporation of liquid mixture to form the discrete particles according to embodiments disclosed herein.

In some embodiments, the electrospray system may include a plurality of coaxially situated hollow capillary tubes (nozzles), each in fluid communication with a pump such that different liquid mixtures may be fed through the nozzles to form discrete particles having a plurality of layers or shells. Suitable examples include electrospray systems having an outer nozzle concentric with an inner nozzle. In some embodiments at least one of the concentric nozzles is not electrically conductive and at least one of the other nozzles, typically at least the innermost nozzle, is electrically conductive. Examples of materials that may comprise insulating non-conductive nozzle include polyether ether ketone (PEEK), polytetrafluoroethylene (PTFE), fluorinated ethylene propylene, high density polyethylene (HDPE), polypropylene, glass, and the like. Materials that may comprise the conductive inner nozzle include stainless steel, aluminum, copper, Hastelloy, gold, platinum, silver, and the like.

In alternative embodiments, two of more of the concentric nozzles are electrically conductive. Accordingly, in some embodiments, all of the nozzles are conductive; in alternative embodiments a non-conductive nozzle may be concentric with a conductive nozzle, and/or a conductive nozzle may be concentric with non-conductive nozzle, and/or a non-conductive nozzle may be concentric with another non-conductive nozzle, or any combination thereof so long as one nozzle is conductive.

The electric potential may be applied to any one or more of the conductive nozzles. The amount of potential may be varied depending on the nozzle, i.e., a first nozzle at a first potential and a second nozzle at a second potential, and/or the potential applied to any one or more nozzles may be varied with time during the spraying process.

In another embodiment, the liquid which passes through one of the nozzles may be a conductive polymer, which transfers charge to another nozzle the liquid is in contact with.

In some embodiments, a first liquid mixture is supplied to a first nozzle and a second liquid mixture is supplied to a second nozzle under electrospraying conditions to form the plurality of particles having a core comprising at least one cannabinoid which is at least partially encapsulated by an outer carrier or “shell” which is then collected on the collection target. By creating core-shell particles encapsulating the cannabinoid, different release profiles may be obtained as the core and one or more shells independently (or not independently) disperse, burst, or otherwise erode in a target environment having certain conditions over a period of time.

In some embodiments, the one or more cannabinoid may be present within any of a plurality of mixtures, dispersions, melts, and/or solutions directed through the electrospray nozzle which is also referred to herein generically as a precursor solution. Accordingly, the cannabinoid or a derivative thereof may be physically dissolved in a solvent and/or dispersed, emulsified or covalently attached to a carrier polymer or biomacromolecule which is then solvated within the precursor solutions prior to fabrication of the particles. Likewise, in the same or a different precursor mixture, the polymeric carrier may be dissolved in a solvent and/or dispersed, emulsified or covalently attached to another carrier polymer or biomacromolecule which is then solvated within the precursor solution prior to fabrication of the particles.

In one or more embodiments, the first precursor solution which forms the core and/or any subsequent precursor solution which forms the “shell” of the core-shell particle comprises the one or more cannabinoids dissolved in an appropriate solvent, preferably this is at least the precursor solution which forms the core of the particle.

In one or more embodiments, the first precursor solution which forms the core and/or any subsequent precursor solution which forms the “shell” of the core-shell particle comprises a hydrophobic polymer such as poly(lactide-co-glycolide) or poly(ε-caprolactone). Suitable polymers for use herein have a molecular weight range from about 200 g/mol to about 5,000,000 g/mol, preferably greater than or equal to about 300 g/mol, or greater than or equal to about 500 g/mol, or greater than or equal to about 1,000 g/mol, or greater than or equal to about 1,500 g/mol, or greater than or equal to about 3,000 g/mol, or greater than or equal to about 5,000 g/mol, or greater than or equal to about 10,000 g/mol, or greater than or equal to about 15,000 g/mol, or greater than or equal to about 20,000 g/mol, and less than or equal to about 4,000,000 g/mol, or less than or equal to about 3,000,000 g/mol, or less than or equal to about 2,000,000 g/mol, or less than or equal to about 1,000,000 g/mol, or less than or equal to about 500,000 g/mol, or less than or equal to about 200,000 g/mol, or less than or equal to about 100,000 g/mol, or less than or equal to about 50,000 g/mol, or less than or equal to about 25,000 g/mol. The concentration may be between about 0.01 wt % to about 1000 wt % depending on the molecular weight of polymer and solvent utilized. Generally, a higher concentration leads to larger-sized particles. The polymer may be dissolved in an appropriate organic solvent including, but not limited to, acetone, dichloromethane, ethyl acetate, chloroform, tetrahydrofuran, dimethyl sulfoxide, trichloroethane, and hexafluoroisopropanol.

In one or more embodiments, the first precursor solution which forms the core and/or any subsequent precursor solution which forms the “shell” of the core-shell particle comprises a hydrophilic polymer such as PEG or PVA.

In one or more embodiments the precursor solution, and thus the final composition further comprises acacia, dextrin, starch, povidone, carboxymethylcellulose, guar gum, glucose, collagen, fish collagen, hydroxypropyl methylcellulose, methylcellulose, hydroxymethylcellulose, polymethacrylates, maltodextrin, hydroxyethyl cellulose, whey, disaccharides, sucrose, lactose, polylactic acid, poly caprylic acid, polyethylene glycol, hypromellose, macrocrystalline cellulose, sorbitol, pectin, or combinations thereof, and/or polysaccharides derivatives thereof and/or ethoxylated and/or propoxylated derivatives thereof. Other suitable polymeric carrier agents are water soluble and include a starch, quillaia extract, maltodextrin, a sugar alcohol, a modified food starch, sorbitol, or a combination thereof.

In other embodiments, the polymeric carrier, is a biocompatible, biodegradable polymer comprising one or more polyester, mixed polyester, polyanhydride, mixed polyanhydride, poly(ester)anhydride, polysaccharide, polyphosphazene or polyphosphoester. Suitable examples include PLGA, polycaprolactone, polylactide, polyglycolide, polyhydroxybutyric acid, poly(sebacic acid), poly[1,6-bis(p-carboxyphenoxy)hexane], and the like.

In one or more embodiments, the carrier, also referred to as the shell of the particles of the composition includes one or more biocompatible, biodegradable polymers selected from poly(ethylene glycols) polyesters, mixed polyesters, for instance PLGA, polyanhydrides, mixed polyanhydrides, poly(ester)anhydrides, polysaccharides, polyphosphazenes, and copolymers and/or combinations thereof.

In one or more embodiments the biocompatible, biodegradable polymer can include one or more of poly(lactic-co-glycolic) acid (“PLGA”), polycaprolactone, polylactide, polyglycolide, polyhydroxybutyric acid, poly(sebacic acid), poly[1,6-bis(p-carboxyphenoxy)hexane], and mixtures thereof. In some embodiments, polycaprolactone (“PCL”) can be used in combination with other polymeric systems such as, for example, poly(ethylene glycols) (“PEG”), and PEG copolymers. Exemplary copolymers include polycaprolactone-poly(ethylene glycol), which may further be appended with a functional group such as an amino, thiol, carboxylate and the like. A preferred biocompatible, biodegradable polymer comprises, consists of, or consists essentially of PCL/PCL-PEG-NH₂.

Suitable polymers for use herein have a molecular weight range from about 200 g/mol to about 5,000,000 g/mol, preferably greater than or equal to about 300 g/mol, or greater than or equal to about 500 g/mol, or greater than or equal to about 1,000 g/mol, or greater than or equal to about 1,500 g/mol, or greater than or equal to about 3,000 g/mol, or greater than or equal to about 5,000 g/mol, or greater than or equal to about 10,000 g/mol, or greater than or equal to about 15,000 g/mol, or greater than or equal to about 20,000 g/mol, and less than or equal to about 4,000,000 g/mol, or less than or equal to about 3,000,000 g/mol, or less than or equal to about 2,000,000 g/mol, or less than or equal to about 1,000,000 g/mol, or less than or equal to about 500,000 g/mol, or less than or equal to about 200,000 g/mol, or less than or equal to about 100,000 g/mol, or less than or equal to about 50,000 g/mol, or less than or equal to about 25,000 g/mol. The concentration may be between about 0.01 wt % to about 1000 wt % depending on the molecular weight of polymer and solvent utilized. Preferably the hydrophilic polymer has a molecular weight range from about 200 g/mol to about 1,500,000 g/mol. The polymer may be dissolved in an appropriated aqueous solvent including, but not limited to, phosphate buffer, Dulbecco's phosphate buffer, HEPES buffer, TRIS buffer, and acetic acid. The viscosity of the precursor solution will be dependent upon the specific material and the solvent in which the material is dissolved. In one or more embodiments, at least one of the precursor solutions comprise a conductive polymer, which is an organic polymer which acts as an electrical conductor or semiconductor. Suitable examples of conductive polymers include polyacetylene, polypyrrole, polyaniline, and derivatives thereof. Additionally, the conductivity of any one or more of the precursor solutions may be increased by the addition of a salt such as sodium chloride, potassium chloride, calcium chloride, magnesium chloride, lithium chloride, sodium carbonate or sodium phosphate, and/or the like. Generally, a more conductive solution will give smaller-sized particles when other electrospray process variables are held constant.

In one or more embodiments, the composition further comprises at least one water and/or water miscible polymeric carrier, preferably selected from complex carbohydrates, polyols, polysaccharides, oligosaccharides, or a combination thereof, wherein the composition is soluble and/or miscible in water at a temperature less than or equal to about 20° C.

In one or more embodiments, the composition further comprises at least one carrier oil, which preferably comprises medium-chain triglyceride (MCT) oil, coconut oil, long-chain triglyceride oil, or a combination thereof. In some embodiments, the cannabinoid mixture may be combined with one or more carrier oils, such as medium chain triglyceride (MCT) oil, long chain triglyceride (LCT) oil, vegetable oil, canola oil, olive oil, sunflower oil, coconut oil (including fractionated coconut oil), hemp oil, palm oils, and/or other oils suitable for consumption. In some cases, the addition of one or more carrier oils may help to improve solubility of the cannabinoid compounds and/or facilitate homogeneous dispersion of the cannabinoid compound(s) into the hydrophilic component or water soluble matrix formed by water and at least one water soluble agent. Further, for example, the carrier oil(s) may be useful to increase the stability of the oil-in-water emulsion, e.g., including for higher levels of cannabinoids. Coconut oil is noted for a high saturated, MCT content. Hemp oil comprises about 80% essential fatty acids and is obtained from hemp seeds, which come from a variety of the Cannabis sativa plant that does not contain a high amount of THC.

If desired, the carrier oil may be purified beforehand, or the combined cannabinoid/carrier oil mixture may be purified according to one or more processes as described above. Together, the carrier oil and the purified cannabinoid mixture may form a hydrophobic component of the composition. In some embodiments, the cannabinoid(s) may be used as a hydrophobic component of the composition with the addition of a carrier oil. In some examples, the weight ratio of carrier oil to cannabinoid mixture (carrier oil:mixture) may range from about 1:100 to about 10:1, such as from about 1:50 to about 5:1, from about 1:10 to about 2:1, from about 3:4 to about 4:3, or from about 1:2 to about 1:1, e.g., a ratio of about 10:1, 5:1, 3:1, 2:1, 4:3, 1:1, 3:4, 1:2, 1:3, 1:5, 1:10, 1:15, 1:20, 1:25, 1:50, 1:75, or 1:100. In some examples, the weight ratio of carrier oil to cannabinoid mixture may range from about 1:4 to about 2:1, from about 1:2 to about 4:3, or from about 1:1 to about 2:1.

In some examples, the composition does not include a carrier oil such as MCT oil, vegetable oil, canola oil, olive oil, sunflower oil, coconut oil, hemp oil, or palm oil. For example, the hydrophobic component of the composition may consistent essentially of, or may consist of, the purified cannabinoid mixture without any other oil(s).

In some embodiments, an excipient may be added to one or more of the precursor solutions to improve release of the cannabinoid or to change the morphology of the particles. Examples of excipients include, but are not limited to, bovine serum albumin (BSA), human serum albumin, trehalose, pluronic surfactants, PEG, PVA, and the like. Pluronics surfactants refer to poly(ethylene oxide)-poly(propylene oxide)-poly(ethylene oxide) triblock copolymers (PEO-PPO-PEO), known in the art for having unique surfactant abilities, low toxicity, and minimal immune response. The concentration of the excipients may be from about 0.01 mg/mL to about 500 mg/mL, depending on the release profile desired to be achieved.

In order to control and/or affect the properties of the particulates formed via the electrospray process, various process conditions in the operation of the electrospray system 200 may be controlled and adjusted. Process conditions which may be adjusted or controlled include, for example, the distance from the tip of the nozzle to the collection target, which may be controlled to affect the wetness or dryness of the core-shell particles. For example, a longer distance may result in drier particles collected as the emulsion droplets emerging from the tubes hit collection target. Likewise, the ambient conditions into which the solutions are sprayed, e.g., temperature, pressure, turbulence, and the like, may be controlled to affect both removal of solvent and/or the formation or lack of formation of agglomerate particles.

Additionally, the inner diameter (ID) of the nozzle tube may be controlled to affect the particle size, and/or the relative inner and outer diameters of concentric tubes may be controlled. Likewise, the application of the electric charge may be applied to one or more of the nozzle tubes and not to others, and/or the voltage applied to the various nozzle tubes may be varied such that each tube has a particular voltage applied. In some embodiments, the voltage applied to any one nozzle tube is varied over time.

In one or more embodiments, the voltage applied to one or more nozzle tubes is in a range from about 0.5 kilovolt (kV) to about 40 kV. Generally, once operating in the critical electrospraying range, higher voltage magnitudes may result in smaller particles until out of the critical range and multi-jetting begins. Additionally, the charge and type of collection targets may be varied to affect yield. For example, in some embodiments, charging collection target with opposite polarity relative to inner tube allows for increased yields. The voltage applied to collection target may be in a range from between about 0.5 kV to about 40 kV. In some embodiments, one or more ring electrodes, referred to as a “third ring electrode” with a polarity that is the same or different from that of the nozzle may allow for better control of spraying by focusing the spray stream for increased particle yield. The one or more ring/third electrodes may be placed proximate to the nozzle, proximate to the collection target, and/or located somewhere in between.

In addition, the absolute flowrates, the relative flowrates, the temperatures and viscosities of the precursor solutions may be selected to control particle size, morphology and/or yield. Typically, the flow rate of the precursor solution may range from about 0.01 milliliters per hour (mL/h) to about 50 mL/h from a single nozzle. A higher flowrate generally results in larger particles. Temperature may range from cryogenic, to sub-ambient to greater than or equal to about 250° C. Generally, a higher temperature will result in higher solvent evaporation and faster processing and may in some cases be utilized when one or more of the precursor solutions is a melt.

In some embodiments, dry collection of the emerging particles from an electrospray system may be employed. The collection target may be comprised of a material that is conductive metal, a non-conductive material with a conductive metal surface, a conductive metal with a non-conductive surface, or an enclosed chamber with turbulent and/or circulating air, such as a cyclone, to stratify the various particles produced. The particles may be sprayed into an inert atmosphere such as nitrogen, argon, into an atmosphere having a post-reaction component such as ammonia, or into the ambient air. The atmosphere may be heated and/or heat may be applied to the collection target to increase solvent evaporation and/or to increase particle yield with dry filter collection.

In other embodiments, wet collection of the emerging particles from an electrospray system may be employed. In this embodiment, collection target is immersed in a liquid bath which may be an aqueous solution and/or a solvent and which may optionally include a surfactant or post reaction component. Examples of suitable surfactants include, but are not limited to, sodium dodecyl sulfate (SDS), tween20, tween80, Pluronic surfactants, PVA, ammonium lauryl sulfate, benzalkonium chloride and other co-polymers of PEO and PPO. Examples of suitable organic solvents include, but are not limited to, ethanol and hexane. In some embodiments, the liquid collection vessel may be agitated and/or sonicated to deagglomerate particles and/or affect the morphology of the collected particles.

Further, according to some aspects of the present disclosure, the precursor mixture may be an emulsion (including, e.g., any of the emulsions described above or elsewhere herein) which is then electrosprayed and dried into particles.

For example, electrospraying into a fluid bed drying apparatus may remove 50% or more of the moisture to leave a particulate composition with 10% or less water moisture by weight, such as from about 0.1% to 10% by weight, from about 0.5% to about 7.5% by weight, from about 1.0% to about 8.0% by weight, from about 1.0% to about 5.0% by weight, or from about 1.5% to about 3.0% by weight water moisture. The particulates then may be separated from the drying air, e.g., based on density or other physical or chemical characteristics, and collected. Electrospray followed by fluid bed drying may be performed as a batch process or a continuous process. The produce may be a flowable powder (e.g., flowable granules) or a compressible powder.

In some cases, it may be desirable for the heated air to use lower drying temperatures and/or shorter drying times to promote greater product stability by reducing oxidative stress and thermal degradation of the components of various actives/bioactives in the composition, including cannabinoid compounds. Lower drying temperatures also may be compatible with a wider range of ingredients, which can be useful for preparing formulations with the appropriate level of water solubility. For example, certain water soluble agents such as sorbitol tend to form a sticky material with poor water solubility at the higher temperatures typical of many fluid bed drying processes.

Accordingly, in some aspects of the present disclosure, drying is performed at a temperature less than or equal to about 80° C., less than or equal to about 70° C., less than or equal to about 60° C., less than or equal to about 50° C., or less than or equal to about 40° C., e.g., a temperature ranging from about 25° C. to about 80° C., from about 30° C. to about 60° C., from about 25° C. to about 50° C., from about 45° C. to about 75° C., or from about 40° C. to about 55° C.

Additionally or alternatively, the residence time in the drying chamber may be less than or equal to 1 hour, less than or equal to 45 minutes, less than or equal to 30 minutes, less than or equal to 20 minutes, less than or equal to 15 minutes, less than or equal to 5 minutes, or less than or equal to 2 minutes, such as from about 5 minutes to about 45 minutes, or from about 20 minutes to about 30 minutes.

In one or more embodiments, the cannabinoid is present in the composition in nanocrystalline form.

In one or more embodiments a method for preparing the composition comprises the steps of: a) providing a first mixture comprising at least one cannabinoid; b) providing a second mixture comprising at least one biocompatible, biodegradable polymer; and c) coaxial electrospinning and/or electrospraying the first and second mixtures onto a collector to form a plurality of discrete particles comprising the at least one cannabinoid at least partially encapsulated by the at least one biocompatible, biodegradable polymer.

In one or more embodiments, the method comprises: providing a solution of the cannabinoid in a solvent; b) electrospraying the solution onto a collector; and c) removing the solvent to produce give the nanocrystalline drug. In embodiments, the solvent is removed by vaporization during the electrospraying. In embodiments the method may further comprise the steps: a) preparing a mixture comprising a nanocrystalline form of a cannabinoid or a derivative thereof, a biocompatible, biodegradable polymer and a water immiscible solvent; and b) combining the mixture with a water miscible solvent under agitation followed by electrospraying to encapsulate the nanocrystalline cannabinoid or derivative thereof, and c) removing the water immiscible solvent; and d) separating encapsulated nanocrystalline drug from the water miscible solvent.

In one or more embodiments the composition is a water-soluble and/or water dispersible powder, a plurality of discrete particles on a surface of a substrate, or a combination thereof.

In one or more embodiments the composition comprises a plurality of discrete particles and/or wherein the composition is in the form of agglomerated particles.

In one or more embodiments, the largest dimension of the discrete particles is less than or equal to about 1 micron and the largest dimension of the agglomerated particles is greater than or equal to about 100 microns, preferably from about 150 microns to about 800 microns.

In embodiments a composition comprising a plurality of discrete particles, each of the particles comprising a cannabinoid at least partially encapsulated within a carrier compositions, the carrier comprising at least one biocompatible, biodegradable polymer, wherein the average longest dimension of the particles is less than or equal to about 1,000 nm, about 900 nm, about 800 nm, about 700 nm, about 600 nm, about 500 nm, about 400 nm, about 300 nm, about 200 nm, or about 100 nm; and wherein the composition comprises greater than or equal to about 5 weight percent of the cannabinoid.

In some embodiments the average longest dimension of the particles is from about 10-1,000 nm, about 10-900 nm, about 10-800 nm, about 10-700 nm, about 10-600 nm, about 10-500 nm, about 10-400 nm, about 10-300 nm, about 10-200 nm, about 10-100 nm, about 100-1,000 nm, about 100-750 nm, about 100-500 nm, 100-400 nm, 100-300 nm, about 100-250 nm, about 100-200 nm, about 50-200 nm, or about 50-100 nm. In embodiments a width of the particle taken perpendicular to the longest dimension of the particles has a standard deviation no greater than 25%, 20%, 10%, 5%, 2.5% or 1% of the average longest dimension.

In embodiments, the discrete particles have a maximum length of less than about 2,500 nm, 1,000 nm, 750 nm, 500 nm, 400 nm, 300 nm, 250 nm, 200 nm, 150 nm, or 100 nm.

In one or more embodiments, the particles of the composition are characterized by a high degree of uniformity, comprising a narrow distribution of their longest dimension, and/or a narrow distribution of particle sizes. The distribution can be characterized by the standard deviation along the longest dimension of the particle of no greater than 25%, 20%, 10%, 5%, 2.5% or 1% of the average.

In one or more embodiments, the particles of the composition are characterized by a high encapsulation efficiency, meaning a minimal amount of the outer carrier composition polymer being present in the core, and/or a minimal amount of the cannabinoid being present in the outer shell of the particle. In some embodiments, the core contains no more than 25%, 20%, 15%, 10%, 7.5%, 5.0%, 2.5%, 1% or 0.5% (w/w) of the outer shell composition, and/or the outer shell or carrier material contains no more than 25%, 20%, 15%, 10%, 7.5%, 5.0%, 2.5%, 1% or 0.5% (w/w) of the cannabinoid.

In one or more embodiments, the particles of the composition provide a relatively high degree of controlled release of the cannabinoid from the carrier or core/shell material. In one or more embodiments, the components of the particles and/or the particles are dimensioned and arranged such that the particles are characterized by the absence of “burst” release of the cannabinoid upon initial exposure to a solvent, e.g., dispersion in water or a beverage, and/or to a biological system after being consumed.

For purposes herein it is assumed that the in vivo release profile can be estimated by measuring release in a system intended to mimic in vivo conditions. For example, immersion of the composition in a buffer solution at a particular temperature, e.g., in 0.01 M PBS (phosphate buffered saline @ pH 7.4; at 37° C., is assumed representative of oral mammalian consumption.

In one or more embodiments, upon oral mammalian consumption of the particles of the composition, no more than 0.1%, 0.5%, 1%, 2%, 3%, 4%, 5%, 6%, 7%, 8%, 9% or 10% of the cannabinoid is released within 5 minutes, or 15 minutes, or 30 minutes, or 1 hour, or 5 hours, or 12 hours, or 24 hours. In one or more embodiments, the composition of the carrier, and/or the relative proportion of the carrier to the cannabinoid of the particles is selected to control the rate of release.

Accordingly, the rate of release can be controlled through proper selection of the biodegradable, biocompatible polymer as well as the relative thickness of the shell material. In some embodiments, at least 50%, 60%, 65%, 70%, 75%, 80%, 85%, 90% or 95% of the cannabinoid is released within a period of 5 minutes, 15 minutes, 30 minutes, 1 hour, 2 hours, 5 hours, or longer, according to a targeted release rate. Likewise, the composition may comprise a plurality of groups of particles, wherein in each group the composition of the carrier, and/or the relative proportion of the carrier to the cannabinoid of the particles is selected to control the rate of release such that a sustained release over a particular period of time is achieved.

In some embodiments, the biocompatible, biodegradable polymer is sufficiently hydrophobic to control the release of the pharmaceutically active agent. The shell polymer can have a contact angle greater than about 90°, or greater than about 100°, or greater than about 110°, or greater than about 120°, or greater than about 130°, or greater than about 140°, or greater than about 150°, or greater than about 160°. In other embodiments, the shell polymer can have a contact angle between about 90-150°, or between about 100-150°, or between about 110-150°, or between about 120-150°, or between about 125-150°. Generally, the core polymer, when present, can be hydrophilic, and can be water soluble such that it degrades/dissolves within 3 hours, within 2 hours, within 1 hour or with 30 minutes of being immersed in water at 20° C. to 25° C.

In one or more embodiments the particles of the composition may be prepared using electrospinning processes. In some embodiments, the particles are in the form of nanofibers prepared using an electrospinning process, whereas discrete particles comprising nanocrystalline forms of the cannabinoids may prepared using an electrospraying process, which may include subsequent electrospray and/or electrospinning processes in which the nanoparticles are further encapsulated with one or more appropriate polymers. Accordingly, in one or more embodiments the output of a first electrospraying process may be used as the feed of a second or subsequent electrospraying process and/or electrospinning process. In other embodiments, nano-encapsulated compositions can be directly prepared using a voltage-switched electrospinning process.

In one or more embodiments, a process to produce the composition comprises the steps of dissolving, mixing or otherwise dispersing the one or more cannabinoids in a first solvent system to produce a first mixture or precursor mixture, and dissolving, mixing or otherwise dispersing the biodegradable, biocompatible polymer in a second solvent system to produce a second precursor mixture. Preferably, the first and second solvents are capable of dissolving the cannabinoid and biodegradable, biocompatible polymer, respectively. In some embodiments, the first and second solvent systems are miscible with each other. In alternative embodiments, the first and second solvent systems are can are immiscible with each other. The solvent systems and by extension the first and/or second mixtures can include other excipients, for instance stabilizers, surfactants, antioxidants, and the like. In some embodiments, the first solvent will not contain any of the biocompatible, biodegradable polymer, and the second solvent will not contain any of the cannabinoid.

Suitable solvents include aprotic solvents including dimethylsulfoxide (DMSO), halogenated hydrocarbons e.g., chloroform, methylene chloride and the like; ethers including dioxane, tetrahydrofuran (THF), dialkyl ethers, e.g., diethyl ether, dimethyl ether, and the like; carbonyl- and/or nitrile-containing compounds including dimethylformamide (DMF), acetone, acetonitrile, ethyl acetate, and the like; can also include protic solvents such as water, organic acids including formic acid, acetic acid, propionic acid, trichloroacetic acid, chloroacetic acid, trifluoroacetic acid and the like, alcohols including methanol, ethanol, ethylene glycol, glycerol, isopropanol, and n-propanol, halogenated alcohols such as 1,1,1,3,3,3-hexafluoro-2-propanol, and the like, and mixtures thereof.

In one or more embodiments the composition further comprises a solvent selected from the group consisting of water, ethanol, DMSO, a vegetable oil, e.g., peanut oil, canola oil, saffron oil, avocado oil, corn oil, and the like, or a combination thereof. Preferably the solvent is a C₂-C₁₀ halogenated alcohol, which in some embodiments is 1,1,1,3,3,3-hexafluoro-2-propanol.

In some embodiments, either the first or second solvent can be a mixture of two or more solvents. In one or more embodiment, the solvent comprises, consists essentially of, or consists of at least one organic acid. In other embodiments, at least one solvent comprises at least one organic acid and a) at least one apolar solvent, b) at least one aprotic solvent, c) at least one protic solvent, or a combination thereof. In one or more embodiments, the ratio (v/v) of organic acid to the remainder of the solvent is from 1:1 to 99:1, 2:1 to 99:1, 3:1 to 99:1, 4:1 to 99:1, 5:1 to 99:1, 7.5:1 to 99:1, 10:1 to 12.5:1, 15:1 to 99:1, or 20:1 to 99:1. In certain embodiments, the ratio (v/v) of organic acid to apolar, and/or aprotic, and/or protic solvent can be at least 85:15, 87.5:1, 90:10, 92.5:7.5, 95:5, 97.2:2.5, 98:2 or 99:1.

Preferred apolar solvents for combination with the organic acid include halogenated hydrocarbons. Preferred protic solvents for combination with the organic acid include alcohols and halogenated alcohols. Preferred organic acids include formic acid, acetic acid, phenol, and mixtures thereof. When the organic acid is a mixture of formic acid and acetic acid, the ratio (v/v) can be from 75:25 to 25:75, 60:40 to 40:60, or 50:50.

In some embodiments in which the first or second solvent include an organic acid as described above, in some embodiments the remaining solvent comprises an aprotic solvent immiscible with the organic acid-containing system. Suitable solvents include DMF, DMSO, methylene chloride, C₅-C₂₀ alkanes like e.g., hexane, cyclohexane, heptane, dodecane, and the like, as well as aromatic hydrocarbons, e.g., toluene, xylene ethyl benzene, and the like.

In one or more embodiments, the cannabinoid is present in the first mixture a concentration from about 1-1000 mg/ml, about 5-500 mg/ml, about 10-100 mg/ml, about 25-100 mg/ml, or about 25-75 mg/ml. The biocompatible, biodegradable polymer can be dissolved in the second solvent at a concentration from about 1-5000 mg/ml, 10-1000 mg/ml, 25-500 mg/ml, 25-400 mg/ml, 25-300 mg/ml, 25-250 mg/ml, 50-250 mg/ml, 100-250 mg/ml, or 100-200 mg/ml.

In one or more embodiments, the composition is produced by a process comprising coaxial electrospraying and/or electrospinning conducted using concentric electrospray needles and/or spinneret nozzles. The mixture comprising the cannabinoid may be dispensed from a needle having a gauge from 15-34, from 15-30, from 20-30, or from 25-30. In some embodiments, the cannabinoid mixture may be sprayed or spun from a needle having a gauge of at least 10, at least 15, at least 20, at least 25, or at least 30. The needle may be placed concentrically into an outer or shell nozzle having an inner diameter that is no more than 0.2 mm, 0.3 mm, 0.4 mm, 0.5 mm, 0.6 mm, 0.7 mm, 0.8 mm, 0.9 mm, 1.0 mm, 1.2 mm, 1.4 mm, 1.6 mm, 1.8 mm or 2.0 mm. In some embodiments, the shell nozzle can have an inner diameter that is at least about 120%, 140%, 160%, 180%, 200%, 250%, 300%, 400%, or 500% the outer diameter of the inner or core needle. In some embodiments, the outer nozzle can have an inner diameter that is between about 120-500%, between about 150-400%, between about 150-300%, or between about 150-250% the outer diameter of the inner needle.

In embodiments, the flow rate of the inner or core mixture through a particular spinneret can be at least 0.05 ml/hr, at least 0.10 ml/hr, at least 0.15 ml/hr, at least 0.20 ml/hr, at least 0.25 ml/hr, at least 0.30 ml/hr, at least 0.35 ml/hr, at least 0.40 ml/hr, at least 0.45 ml/hr, or at least 0.50 ml/hr. The flow rate of the inner or core solution through the spinneret can be between 0.05 ml/hr and 0.50 ml/hr, between 0.05 ml/hr and 0.40 ml/hr, between 0.05 ml/hr and 0.30 ml/hr, between 0.10 ml/hr and 0.30 ml/hr, or between 0.20 ml/hr and 0.30 ml/hr.

In one or more embodiments, the flow rate of the outer or shell mixture solution through the spinneret can be at least 0.10 ml/hr, at least 0.20 ml/hr, at least 0.30 ml/hr, at least 0.40 ml/hr, at least 0.50 ml/hr, at least 0.60 ml/hr, at least 0.70 ml/hr, at least 0.80 ml/hr, at least 1.0 ml/hr, at least 1.25 ml/hr, or at least 1.50 ml/hr. The flow rate of the core solution through the spinneret can be between 0.10 ml/hr and 1.50 mg/hr, between 0.10 ml/hr and 1.0 ml/hr, between 0.20 ml/hr and 1.0 ml/hr, between 0.10 ml/hr and 0.50 ml/hr, or between 0.25 ml/hr and 0.75 ml/hr.

In one or more embodiments, the applied voltage for the electrospinning and/or electrospraying is greater than about 1 kV, preferably greater than about 5 kV, preferably greater than about 10 kV, preferably greater than about 50 kV, preferably greater than about 75 kV, and preferably less than about 100 kV, preferably less than about 75 kV, preferably less than about 50 kV, preferably less than about 10 kV, preferably between 1-100 KV, between 10-100 KV, between 10-75 KV, between 10-50 KV, between 10-40 KV, between 15-40 KV, between 15-30 KV, or between 15-25 KV.

The distance from tip to a collector can be at least 50 mm, at least 75 mm, at least 100 mm, at least 125 mm, at least 150 mm, at least 175 mm, at least 200 mm, at least 250 mm, or at least 300 mm. In some embodiments, the distance from tip to collector can be from 50-300 mm, from 75-250 mm, from 100-250 mm, or from 100-200 mm. After electrospinning and/or electrospraying, the collected fibers and/or particles of the composition can be washed in an appropriate solvent to remove residual cannabinoids and/or other materials present on the surface of the fibers and/or particles.

Depending on the cannabinoid and/or the derivative or other analog of the cannabinoid used, embodiments comprising nanocrystalline particles of the cannabinoid may be obtained by an electrospraying process. Typically, the cannabinoid to be crystallized is dissolved in one or more of the solvents described herein, and the material is electrosprayed according to conditions disclosed herein onto a substrate. The nanocrystalline particles may then be collected from the substrate and/or may be directed into a second or subsequent mixture for additional processing including further electrospraying and/or electrospinning.

In one or more embodiments, the particles of the composition may form agglomerates during the process. In one or more embodiments, these agglomerates may be further reduced in size using physical agitation, for instance, sonication, fluidized bed drying, and/or the like. One or more immiscible solvents may also be added to further reduce the particle size of the particulates via sonication. In one or more embodiments, the particles produced by the electrospraying process are directed onto a collector which then serves as the vessel for sonication.

In one or more embodiments according to the present disclosure, a composition comprises a plurality of discrete particles comprising one or more cannabinoids at least partially disposed within and/or on a polymeric carrier produced via electrospraying deposition pf a precursor mixture, wherein each of said discrete particles have a maximum dimension of less than or equal to about 1 micron. Preferably at least a portion of the polymeric carrier is water soluble and/or water miscible.

In one or more embodiments of the composition comprise a plurality of nanofibers comprising one or more cannabinoids at least partially disposed within and/or on a polymeric carrier produced via electrospinning deposition of a precursor mixture. Preferably, the polymeric carriers are water soluble and/or water miscible.

For ease of disclosure, the discrete particles comprising the cannabinol are described in terms of a core and a shell. However, it is to be understood that in addition to the particle core, which preferably comprises the at least one cannabinol and/or a derivative thereof, the particle may comprise a plurality of shell layers which at least partially encapsulate the core. Furthermore, one or more of the shell layers may further include at least one cannabinol and/or a derivative thereof, which may be the same or may be different than that may be present in the core. Likewise, the composition of each shell may be the same or different than another shell and/or the thickness of each shell may be the same or different than another present in the same particle.

In embodiments, both the core and shell materials may include a material that is thermoplastic, biocompatible and bioerodable. “Thermoplastic” is a property wherein the material is soft and pliable when heated. As discussed elsewhere herein, “biocompatible” means that the material has the capability of co-existing with living tissues or organisms without causing substantial harm. “Bioerodable” means that the material has the capability to degrade over time under physiological conditions. Examples of such materials include, but are not limited to, polymers and biomacromolecules. In some embodiments, the shell material(s) may be overall hydrophobic materials, while the core material may be overall hydrophilic materials. In other embodiments, the shell material(s) may be overall hydrophilic materials, while the core material may be overall hydrophobic materials.

Examples of suitable hydrophobic and hydrophilic polymers include, but are not limited to, polypropylene; polypropyleneglycol (PPG); polyvinylpyrrolidone (PVP); poly(ester amide) (PEA); acrylic acid (AA); polyacrylates such as poly(methyl methacrylate) (PMMA), poly(butyl methacrylate), poly(ethyl methacrylate), hydroxyethylmethacrylate (HEMA), poly(ethyl methacrylate-co-butyl methacrylate) (P(MMA-co BMA)), ethyl glycol dimethacrylate, (EGDMA) and ethylene-methyl methacrylate copolymers; acrylamides such as N,N-dimethyl acrylamide, diacetone acrylamide, and acrylamide-methyl-propane sulfonate (AMPS); fluorinated polymers or copolymers such as poly(vinylidene fluoride) and poly(vinylidene fluoride-co-hexafluoro propene); poly(N-vinyl pyrrolidone); poly(N-vinyl pyrrolidone-co-vinyl acetate); poly(hydroxyvalerate); poly(L-lactic acid)/polylactide (PLLA); poly(.epsilon.-caprolactone); poly(L-lactide-co-caprolactone); poly(lactide-co-glycolide) (PLGA); poly(hydroxybutyrate); poly(hydroxyvalerate); poly(hydroxybutyrate-co-valerate); polydioxanone; polyorthoester; polyanhydride; poly(glycolic acid)/polyglycolide (PGA); poly(D,L-lactic acid) (PLA); poly(glycolic acid-co-trimethylene carbonate); polyphosphoester; polyurethanes such as polyphosphoester urethane, poly(amino acids); cyanoacrylates; poly(trimethylene carbonate); poly(iminocarbonate); co-poly(ether-esters); polyalkylene oxalates; polyphosphazenes; silicones; polyesters; polyolefins; polyisobutylene and ethylene-alphaolefin copolymers; vinyl halide polymers and copolymers such as polyvinyl chloride (PVC); polyvinyl ethers such as polyvinyl methyl ether; polyvinylidene chloride; polyacrylonitrile; polyvinyl ketones; polyvinyl aromatics such as polystyrene, styrene sulfonate and acrylonitrile-styrene copolymers; polyvinyl esters such as polyvinyl acetate; copolymers of vinyl monomers with each other such as divinyl benzene (PVB); olefins such as poly(ethylene-co-vinyl alcohol) (EVAL); acrylonitrile butadiene (ABS) resins; and ethylene-vinyl acetate copolymers; polyamides such as Nylon 66 and polycaprolactam; alkyd resins; polycarbonates; polyoxymethylenes; polyimides; polyethers; epoxy resins; polyurethanes polyurethane(ureas); biodegradable polyurethanes; biodegradable polyurethane(ureas); rayon; and rayon-triacetate, poly(ethylene glycol) (PEG), and poly(vinyl alcohol) (PVA).

In embodiments, suitable biomacromolecule may include, but are not limited to, fibrin; fibrinogen; dextran; cellulose including cellulose, cellulose acetate, cellulose butyrate, cellulose acetate butyrate, cellophane, cellulose nitrate, cellulose propionate, cellulose ethers, and carboxymethyl cellulose; starch; pectin; chitosan; gelatin; alginate and conjugations thereof including alginate-gelatin, alginate-collagen, alginate-laminin, alginate-elastin, alginate-collagen-laminin and alginate-hyaluronic acid; collagen and conjugates thereof; hyaluronan; hyaluronic acid; sodium hyaluronate; modified hyaluronan such as tyramine-hyaluronate or glycidyl methacrylate hyaluronate; or self-assembled peptides including dipeptides, so called “lego peptides, ionic self-complementary peptides, surfactant peptides, molecular paint peptides, carpet peptides, cyclic peptides, and the like.

In some embodiments the macromolecule may comprise carbon nanotubes.

Preferably, the discrete particles according to the instant disclosure have an average particle size of less than about 1000 nm, or 500 nm, or 250 nm, or 200 nm, or 150 nm, or 100 nm, or 75 nm, or 50 nm, or 25 nm, or 10 nm, or less than 5 nm.

In one or more embodiments, the particulates produced by a first electrospraying process may subsequently be used as the feed for a second electrospraying process such that various encapsulation layers are present, each

In one or more embodiments of the composition, the one or more cannabinoids are present in the composition in an amount from about 0.1 wt % to about 50 wt %, based on the total weight of the composition present. In one or more embodiments, the one or more cannabinoids comprise cannabidiol or a derivative thereof. In other embodiments, the one or more cannabinoids consist essentially of, or consist of cannabidiol or a derivative thereof.

### Cannabinoids

Suitable cannabinoids for use herein include both optically pure and racemic pairs of compounds which may be isolated from one or more of the Cannabis sativa plants including chemotypes I, II, III, and the like. Suitable cannabinoids for purposes herein may be isolated from the Cannabis sativa plant and/or may be synthetically produced and/or modified, and/or biosynthesized. Unless explicitly stated otherwise, the term “cannabinoids” refers to one or more of the cyclized and/or uncyclized, substituted and/or unsubstituted forms of:

i) cannabigerol, according to the general formula:

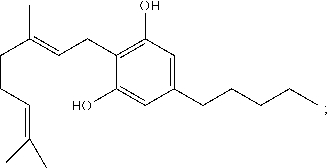

ii) cannabichromene, according to the general formula:

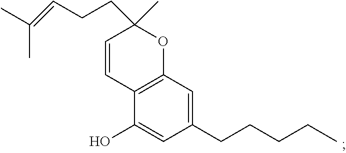

iii) cannabidiol, according to the general formula:

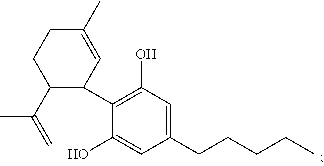

iv) tetrahydrocannabinol and/or cannabinol, according to the general formula:

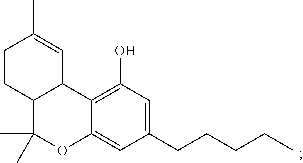

v) cannabielsoin, according to the general formula:

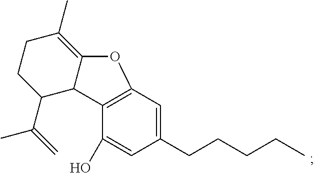

vi) iso-tetrahydrocannabinol, according to the general formula:

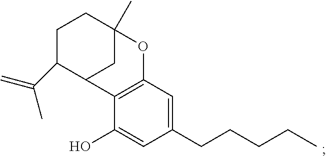

vii) cannabicyclol, according to the general formula:

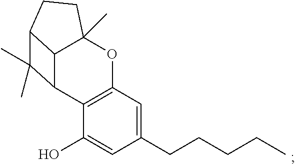

viii) cannabicitran, according to the general formula:

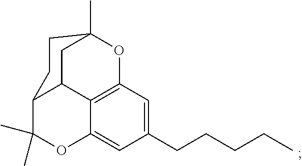

and/or
ix) tetrahydrocannabivarin (THCV), according to the general formula:

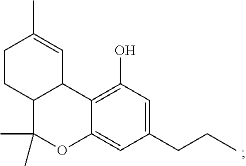

wherein any one or more of the various hydrogen atoms may be substituted with a functional group, and/or including the free acids, salts, tosylates, mesylates, esters, amides, ethers, sulfates, and/or other derivatives thereof.

Specific examples of cannabinoids include the various tetrahydrocannabinols (THC) in general, and (−)-trans-Δ⁹-tetrahydrocannabinol in particular, cannabidol (CBD), tetrahydrocannabinolic acid (THCA), cannabidiolic acid (CBDA), cannabinol (CBN), cannabigerol (CBG), cannabichromene (CBC) cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), cannabigerolic acid, cannabigerolic acid monomethylether, cannabigerol monomethylether, cannabigerovarinic acid, cannabichromenic acid, cannabichromevarinic acid, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-isotetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxycannabitriolvarin, dehydrocannabifuran, cannabifuran, cannabichromanon, cannabicitran, 10-oxo-delta-6a-tetrahydrocannabinol, delta-9-cistetrahydrocannabinol, 3, 4, 5, 6-tetrahydro-7-hydroxy-alpha-alpha-2-trimethyl-9-npropyl-2, 6-methano-2H-1-benzoxocin-5-methanol-cannabiripsol, trihydroxy-delta-9-tetrahydrocannabinol, cannabinol, and/or derivatives thereof.

The cannabinoids may be isolated from plants, e.g., Cannabis sativa, and/or may be produced synthetically, and/or may be isolated from plants and subsequently modified via natural and/or synthetic means, and/or derivatized according to one or more embodiments disclosed herein.

In one or more embodiments, the phenolic hydrogen, when present, is replaced by a C₁-C₄₀ hydrocarbyl, preferably a C₃-C₄₀ carbohydrate, saccharide or polysaccharide, optionally comprising one or more functional groups, e.g., an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more embodiments, the cannabinoid comprises both substituted and unsubstituted forms of cannabidiol (CBD) according to the general formula:

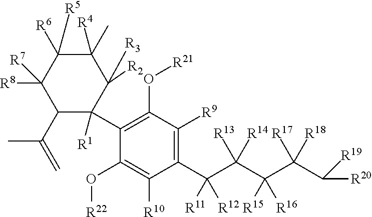

wherein one or more of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², are independently selected from hydrogen or one or more monovalent radicals including hydrocarbyl radicals such as methyl, ethyl, ethenyl, and all isomers (including cyclics such as cyclohexyl) of propyl, butyl, pentyl, hexyl, heptyl, octyl, nonyl, decyl, undecyl, dodecyl, propenyl, butenyl, and from halocarbyls and all isomers of halocarbyls including perfluoropropyl, perfluorobutyl, perfluoroethyl, perfluoromethyl, and from substituted hydrocarbyl radicals and all isomers of substituted hydrocarbyl radicals including trimethylsilylpropyl, trimethylsilylmethyl, trimethylsilylethyl, and from phenyl, and all isomers of hydrocarbyl substituted phenyl including methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, dipropylmethylphenyl, and the like; from all isomers of halo substituted phenyl (where halo is, independently, fluoro, chloro, bromo and iodo) including halophenyl, dihalophenyl, trihalophenyl, tetrahalophenyl, and pentahalophenyl; and from all isomers of halo substituted hydrocarbyl substituted phenyl (where halo is, independently, fluoro, chloro, bromo and iodo) including halomethylphenyl, dihalomethylphenyl, (trifluoromethyl)phenyl, bis(triflouromethyl)phenyl; and from all isomers of benzyl, and all isomers of hydrocarbyl substituted benzyl including methylbenzyl, dimethylbenzyl.

In one or more embodiments, R²¹ and/or R²², comprise a C₃-C₄₀ carbohydrate, saccharide or polysaccharide, optionally comprising one or more functional groups, e.g., an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In some embodiments, one or more of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², is substituted with one or more functional groups selected from Br, Cl, F, I, —NR*2, —NR*—CO—R*, —OR*, *—O—CO—R*, —CO—O—R*, —SeR*, —TeR*, —PR*2, —PO—(OR*)₂, —O—PO—(OR*)₂, —AsR*2, —SbR*2, —SR*, —SO₂—(OR*)₂, —BR*2, —SiR*3, —GeR*3, —SnR*3, —PbR*3, —(CH₂)q-SiR*3, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

In one or more embodiments, R²⁰ and/or R²¹ is substituted with monovalent functional group comprising a triose, a tetrose, a pentose, a hexose, a heptose, a glycoside, and/or a combination thereof.

In embodiments, the compositions according to the instant disclosure may further comprise one or more surfactants and/or a surfactant system. In one or more embodiments the composition further comprises one or more surfactants and/or a surfactant system. In some embodiments, the surfactant is a phospholipid, a sugar fatty acid ester, a sucrose fatty acid ester, a polysorbate and a polysorbate analog, or a combination thereof. In one or more embodiments of the composition, at least one surfactant has an HLB of greater than or equal to about 10 and/or further comprises at least one surfactant having an HLB of less than 10. In one or more embodiments the surfactant system comprises a matched pair suitable for aqueous dispersion.

In one or more embodiments the composition further comprises an emulsion stabilizer selected from the group consisting of xanthan gum, guar gum and sodium alginate; modified gum acacia; ester gum, or a combination thereof, and/or a pH adjuster present in an amount sufficient to adjust the pH of 1 wt % of the composition in deionized water to greater than or equal to about 6 and less than or equal to about 8 at 25° C.

The composition may further comprise a hydrophilic component, e.g., comprising one or more water soluble agents. Exemplary water soluble agents include, but are not limited to, carbohydrates, including complex carbohydrates such as starches, gum arabic, and quillaja extract; sugars such as monosaccharides (e.g., dextrose), oligosaccharides (e.g., cyclodextrins), and polysaccharides (e.g., maltodextrin); and polyols including, e.g., sugar alcohols such as sorbitol and maltitol. Additional water soluble agents that may be used herein include proteins (e.g., gelatin, whey, casein), phospholipids (e.g., soy lecithin, egg lecithin, etc.), glycerol monostearate, surfactants (such as, e.g., sorbitan, sorbitan esters, and polysorbates (e.g., sorbitan monolaurate, polyoxyethylene (20) sorbitan monolaurate, sorbitan monopalmitate, polyoxyethylene (20) sorbitan monopalmitate, sorbitan monostearate, polyoxyethylene (20) sorbitan monostearate, sorbitan tristearate, sorbitan monooleate, polyoxyethylene (20) sorbitan monooleate, etc.), and other emulsifiers and water soluble agents suitable for human consumption. The water soluble agent(s) may have a chemical structure that includes a hydrophilic region to promote solubility. Without intending to be bound by theory, it is believed that the water soluble agent(s) may promote solubility of the cannabinoid compounds, e.g., by at least partially absorbing the cannabinoid compounds or otherwise associating the cannabinoid compounds with hydrophilic portions of the water soluble agent.

Examples of water soluble agents include those comprising one or more complex carbohydrates, including e.g., natural carbohydrates such as starches, gum arabic, and quillaja extract. The starch may be a food starch (e.g., waxy maize, corn, potato, wheat, tapioca, or cassava, etc.), and may be relatively high in amylopectin and/or chemically modified to increase an oil absorption capacity of the starch. Examples of starches suitable for the compositions herein include different types of modified food starches, including, but not limited to, octenyl succinic anhydride (OSA) starch. In some examples, the composition may comprise at least one complex carbohydrate in combination with one or more other water soluble agents, such as, e.g., oligosaccharides, polysaccharides, surfactants, and/or polyols. Further, for example, the composition may comprise two or more different complex carbohydrates, optionally in combination with one or more oligosaccharides, polysaccharides, surfactants, and/or polyols. Commercial examples of water soluble agents suitable for the compositions and methods herein include, but are not limited to, CAPSUL®. starch, PURITY GUM® starch, N-ZORBIT® starch, PENBIND® starch, N-Lite® LP starch, and Q-Naturale® quillaja extract produced by Ingredion; and Span® 20, Span® 40, Span® 60, Span® 80, Tween® 20, Tween® 40, Tween® 60, and Tween® 80, produced by Croda International PLC.

In some embodiments, the composition comprises at least one water soluble agent chosen from a complex carbohydrate, a polyol, a polysaccharide, an oligosaccharide, or a combination thereof. For example, the water soluble agent(s) may comprise a starch, quillaja extract, maltodextrin, a sugar alcohol, or a combination thereof. In at least one example, the water soluble agent(s) comprise a modified food starch, sorbitol, or both. According to some aspects of the present disclosure, the composition comprises at least two water soluble agents. For example, the composition may comprise two or more different water soluble agents chosen from complex carbohydrates, polyols, polysaccharides, oligosaccharides, and combinations thereof. Further, for example, the two or more different water soluble agents may be chosen from modified food starches, sugar alcohols, quillaja extract, maltodextrin, or combinations thereof. In some examples, the two different water soluble agents comprise a starch and a sugar alcohol.

Certain water soluble agent(s) may provide sweetness to the composition. For example, sorbitol is a sugar alcohol that is generally understood to be metabolized at a slower rate than sugar, and thus may be described as a sugar substitute. Further, for example, maltodextrin is a long-chain polysaccharide that may be described as moderately sweet. In general, a longer chain length corresponds to a composition with less sweetness. For example, the water soluble agent(s) may comprise a polysaccharide or oligosaccharide that does not provide any sweetness, e.g., a polysaccharide or oligosaccharide that is flavorless.

In some examples herein, the weight ratio of water soluble agent(s) to hydrophobic component (i.e., purified oil distillate and carrier oil(s), if any) may range from about 10:1 to about 1:100, such as from about 5:1 to about 1:50, from about 4:1 to about 1:20, from about 3:1 to about 1:15, from about 2:1 to about 1:10, or from about 4:1 to about 1:4, e.g., a ratio of about 10:1, 5:1, 4:1, 3:1, 5:2, 2:1, 4:3, 1:1, 3:4, 1:2, 2:5, 1:3, 1:4, 1:5, 1:10, 1:25, 1:50, 1:75, or 1:100. In some examples, the weight ratio of water soluble agent(s) to the hydrophobic component ranges from about 1:5 to about 2:1, e.g., a weight ratio of up to about 1:1, up to about 1:2, up to about 1:3, up to about 1:4, or up to about 1:5.

In embodiments, the surfactant is a phospholipid, a sugar fatty acid ester, a sucrose fatty acid ester, a polysorbate and a polysorbate analog, or a combination thereof. In some embodiments, at least one surfactant has an HLB of greater than or equal to about 10 and/or may further comprise at least one surfactant having an HLB of less than 10.

The compositions may further include one or more other or co-surfactants to improve emulsification of the cannabinoid and/or the stability of the composition, for example, by preventing or slowing oxidation of the cannabinoid or other ingredient.

Suitable surfactants include phospholipids, for example, phosphatidylcholine. Other exemplary surfactants include non-ionic surfactants, such as sugar-derived surfactants, including fatty acid esters of sugars and sugar derivatives, and PEG-derived surfactants, such as PEG derivatives of sterols, PEG derivatives of fat-soluble vitamins and PEG-sorbitan fatty acid esters. Polyethylene/polypropylene/polybutene glycols may also be used.

When present, the amount of the surfactant is typically less than or equal to about 10 wt %, typically less than or less than about 5%, for example, the total amount of surfactant as a percentage (%), by weight, of the composition can be, e.g., less than or less than about 10%, such as less than or about 5%, 4.5%, 4%, 3.5%, 3.15%, 3%, 2.5%, 2%, 1.75%, 1.5%, 1.25%, 1%, 0.75%, 0.5%, 0.25%, 0.15% or less, by weight, of the total composition.

Suitable phospholipids include, but are not limited to lecithin, including phosphatidylcholine (PC), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM) or a combination thereof. Typically, the phospholipid is phosphatidylcholine (PC), which sometimes is referred to by the general name “lecithin.” Exemplary of the phospholipids that can be used as co-surfactants in the provided compositions are the phospholipids sold by Lipoid, LLC (Newark, N.J.), for example, Purified Egg Lecithins, Purified Soybean Lecithins, Hydrogenated Egg and Soybean Lecithins, Egg Phospholipids, Soybean Phospholipids, Hydrogenated Egg and Soybean Phospholipids, Synthetic Phospholipids, PEG-ylated Phospholipids and phospholipid blends. Exemplary of the phosphatidylcholine that can be used as a co-surfactant in the provided compositions is the phosphatidylcholine composition sold by Lipoid, LLC, under the name Lipoid S100, which is derived from soy extract and contains greater than or greater than about 95% phosphatidylcholine.

Suitable sugar-derived surfactants include, but are not limited to, sugar fatty acid esters including fatty acid esters of sucrose, glucose, maltose and other sugars, esterified to fatty acids of varying lengths (e.g., containing a varying numbers of carbons). The fatty acids typically have carbon chains between 8 and 28 carbons in length, and typically between 8 and 20, or between 8 and 18 or between 12 and 18, such as, but not limited to, stearic acid (18 carbons), oleic acid (18 carbons), palmitic acid (16 carbons), myristic acid (14 carbons) and lauric acid (12 carbons). Typically, the sugar ester surfactants are sucrose ester surfactants, typically sucrose fatty acid ester surfactants.

Suitable polyalkylene derived surfactants include, but are not limited to PEG derivatives of sterols, e.g., a cholesterol or a sitosterol (including, for example, any of the PEG derivatives disclosed in U.S. Pat. No. 6,632,443); PEG derivatives of fat-soluble vitamins, for example, some forms of vitamin A (e.g., retinol) or vitamin D (e.g., vitamin D1-D5); and PEG-sorbitan fatty acid esters, such as polysorbates, including polyoxyethylene (20) sorbitan monooleate (also called polysorbate 80) and analogs (e.g., homologs) of polysorbate 80, such as, for example, polysorbate 20 (polyoxyethylene (20) sorbitan monolaurate), polysorbate 40 (polyoxyethylene (20) sorbitan monopalmitate) and polysorbate 60 (polyoxyethylene (20) sorbitan monostearate); and stearic acid derivatives, including, for example, polyethylene glycol 400 distearate (PEG 400 DS), such as the PEG 400 DS sold by Stepan Lipid Nutrition (Maywood, N.J.).

Suitable sugar fatty acid ester surfactants include sucrose fatty acid esters wherein the fatty acid contains between 4 and 28 carbon atoms, typically between 8 and 28 carbon atoms, and typically between 8 and 25 carbon atoms, such as between 8 and 18 carbon atoms, such as 8, 9, 10, 11, 12, 13, 14, 15, 16, 17 and 18 carbon atoms. The fatty acid can be synthetic or naturally occurring and include linear and branched fatty acids. The fatty acids include, but are not limited to, myristic acid, palmitic acid, stearic acid, oleic acid, caproic acid, capric (or decanoic) acid, lauric acid, caprylic acid and pelargonic (or nonanoic) acid.

In embodiments, the sugar fatty acid ester is a sucrose fatty acid ester surfactant which may be sucrose monoesters, diesters, triesters and polyesters, and mixtures thereof, and typically contain sucrose monoesters. The sucrose fatty acid ester surfactants include single fatty acid esters and also include homogeneous mixtures of sucrose esters, containing members with different lengths of fatty acid carbon chain and/or members with different degrees of esterification. For example, the sucrose fatty acid ester surfactants include mixtures of monoesters, diesters, triesters, and/or polyesters. The sugar ester surfactants further include sucrose fatty acid ester analogs and homologs and mixtures thereof.

Suitable sucrose fatty acid esters include mixtures of sucrose fatty acid esters, and may have varying HLB values, such as HLB values ranging from at or about 1 to at or about 20. The HLB value of the sucrose fatty acid ester generally depends on the degree of esterification (e.g., the average degree of esterification in a mixture of different esters). Typically, the lower the degree of esterification (e.g., average degree), the higher the HLB value of the sucrose fatty acid ester or mixture thereof. Exemplary sucrose esters include sucrose distearate (HLB=3), sucrose distearate/monostearate (HLB 12), sucrose dipalmitate (HLB=7.4), sucrose monostearate (HLB=15), sucrose monopalmitate (HLB>10), sucrose monolaurate (HLB 15). Typically, the sucrose fatty acid ester surfactants in embodiments of the particulate composition have an HLB value of between at or about 13 and at or about 20, such as at or about 13, 14, 15, 16, 17, 18, 19, or 20, and typically between at or about 13 and at or about 18, such as, but not limited to, HLB values of at or about 15, 16 and 17, such as, for example, sucrose ester surfactants including sucrose monopalmitate, sucrose monolaurate and sucrose monostearate.

In embodiments the sucrose ester mixtures have at least at or about 50%, by weight (w/w), monoester, such as at least or about at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%, by weight (w/w), sucrose monoesters, and typically at least at or about 60%, by weight, or at least at or about 70%, by weight (w/w), monoesters.

Suitable examples of sucrose fatty acid ester surfactants include sucrose fatty acid monoesters, such as sucrose monocaprylate, sucrose monodecanoate, sucrose monolaurate, sucrose monomyristate, sucrose monopalmitate, sucrose monostearate, sucrose monopelargonate, sucrose monoundecanoate, sucrose monotridecanoate, sucrose monopentadecanoate and sucrose monoheptadecanoate. The sucrose fatty acid esters further include mixtures containing varying percentages of monoesters, diesters, triesters and polyesters, such as, but not limited to, a mixture having at or about 72% monoesters, 23% diesters, 5% triesters and 0 polyesters; a mixture having at or about 61% monoesters, 30% diesters, 7% triesters, and 2% polyesters; and a mixture having at or about 52% monoesters, 36% diesters, 10% triesters and 2% polyesters.

In embodiments, the composition further includes one or more emulsion stabilizers (co-emulsifiers), which can be used to stabilize the liquid nanoemulsion upon dilution of the composition into an aqueous solvent. In embodiments, the emulsion stabilizer functions to increase the viscosity of embodiments of the particulate composition or the mixture produced by dilution thereof.

Exemplary of an emulsion stabilizer that can be used in the provided compositions is a composition containing a blend of gums, for example, gums used as emulsifying agents, for example, a blend containing one or more of xanthan gum, guar gum and sodium alginate. Exemplary of such an emulsion stabilizer includes the emulsion stabilizer sold under the brand name SALADIZER®, available from TIC Gums, Inc. (Belcamp, Md.). Other gums can be included in the emulsion stabilizer, for example, gum acacia, ester gums and sugar beet pectin. Exemplary emulsion stabilizers include modified food starches. These include the modified gum acacia sold under the name Tic Pretested® Ticamulsion A-2010 Powder, available from TIC Gums, Inc. (Belcamp, Md.). Other exemplary emulsion stabilizers containing an ester gum are, for example, the emulsion stabilizer sold under the name Tic Pretested® Ester Gum 8BG, available from TIC Gums, Inc. (Belcamp, Md.) or Ester Gum 8BG, available from Hercules/Pinova (Brunswick, Ga.). Others sold by Ingredion, Inc (Westchester, Ill.) under the trademarks CAPSUL®, FIRMTEX®, THERMFLO®, THERMTEX®, and TEXTRA® and others, can be included in the compositions provided herein. Other blends of similar gums can also be used as emulsion stabilizers.

The emulsion stabilizer can be added to the water phase, the oil phase, or both the water and the oil phase, during formation of the particulates. In embodiments, the emulsion stabilizer is present in the composition at greater than or equal to about 0.1 wt % or about 0.1% and 1% or about 1%, for example, 0.1%, 0.12%, 0.13%, 0.14%, 0.15%, 0.16%, 0.17%, 0.18%, 0.19%, 0.2%, 0.25%, 0.3%, 0.31%, 0.32%, 0.33%, 0.34%, 0.35%, 0.36%, 0.37%, 0.38%, 0.39%, 0.4%, 0.5%, 0.6%, 0.7%, 0.8%, 0.9% or 1 wt %, or 5%, 10%, 15%, 18%, 20%, or 25%, by weight, or more.

The particulate composition may further include one or more flavoring agents, for example, any compound that can add flavor upon dilution into an aqueous liquid. Exemplary of flavors that can be used are fruit flavors, such as guava, kiwi, peach, mango, papaya, pineapple, banana, strawberry, raspberry, blueberry, orange, grapefruit, tangerine, lemon, lime and lemon-lime; cola flavors, tea flavors, coffee flavors, chocolate flavors, dairy flavors, root beer and birch beer flavors, methyl salicylate (wintergreen oil, sweet birch oil), citrus oils and other flavors. Typically, the flavors are safe and/or desirable for human consumption, for example, GRAS or Kosher-certified flavors. An exemplary flavoring agent that can be used in embodiments of the particulate composition include lemon oil, for example lemon oil sold by Mission Flavors (Foothill Ranch, Calif.), and D-limonene, for example, 99% GRAS certified D-Limonene, sold by Florida Chemical (Winter Haven, Fla.).

In embodiments, the particulate composition further includes one or more pH adjusters which may be added at an appropriate concentration to achieve a desired pH. Suitable pH adjuster are added to adjust the pH of the mixture produced upon dilution of the particulates in water to a pH of greater than or equal to about 2 to less than or equal to about 9, or from about 2 to 8, or 5 to 7.5, or from 3 to 4.0 or 4 to 6. In embodiments, the pH adjuster present in an amount sufficient to adjust the pH of 1 wt % of the composition in deionized water to greater than or equal to about 6 and less than or equal to about 8 at 25° C.

One or more of a plurality of pH adjusting agents can be used. Typically, the pH adjusting agent is safe for human consumption, for example, GRAS certified. The pH adjuster can be citric acid. An exemplary pH adjuster suitable for use with embodiments of the particulate composition includes the citric acid sold by Mitsubishi Chemical (Dublin, Ohio). Another exemplary pH adjuster is phosphoric acid, such as Food Grade 80% Phosphoric Acid, sold by Univar. Various buffer compositions may also be employed.

Typically, the concentration of pH adjuster added according to embodiments of the particulate composition at less than 5% or about 5%, for example, less than or about 4%, 3.5%, 3%, 2.5%, 2%, 1.5%, 1%, 0.9%, 0.8%, 0.7%, 0.6%, 0.5%, 0.4%, 0.3%, 0.2%, 0.1% or less, by weight, of the particulate composition.

In embodiments the particulate composition my further include various other components such as soluble fiber. Soluble fibers include any soluble dietary fiber that can be readily fermented in the colon, typically a plant based dietary fiber, for example, a soluble fiber from legumes, vegetables, such as broccoli and carrots, root vegetables, such as potatoes, sweet potatoes and onions, oats, rye, chia, barley and fruits, such as prunes, plums, berries, bananas, apples and pears. Typically, soluble dietary fiber contains non-starch polysaccharides, such as arabinoxylans, cellulose, dextrans, inulin, beta-glucans, fructo-oligosaccharides, oligosaccharides and polysaccharides. Soluble fibers include, but are not limited to, fructo-oligosaccharides, for example, inulins, for example, inulins found in chicory, Jerusalem artichoke, dahlia, garlic, leeks and onions, fructans and water-soluble soybean fiber. Exemplary of a soluble fiber is an inulin, for example, Oliggo-Fiber Instant Inulin (Fibruline® Instant) (supplied by Cosucra-Groupe Warcoing SA, Belgium, sold by Gillco Products, San Marcos, Calif.), containing chicory inulin. Such materials may be a substrate onto which the discrete particles of the electrosprayed composition are disposed.

Other additional components include sweeteners, glidents, anti-caking agents, antifoaming agents, and the like.

In one or more embodiments, the particulate composition my further include one or more stabilizers, or a stabilizing system. Stabilizers include any compound used to stabilize the cannabinoids and/or other non-polar ingredients in the particulate composition, and/or upon dilution of the particulate composition in an aqueous solvent. Suitable stabilizer or stabilizing systems include, but are not limited to, carbonates and bicarbonates, acids, antioxidants, and any combination thereof. Typically, the stabilizers or stabilizing system are food-approved, i.e., edible or ingestible, stabilizers, for example, stabilizers that are safe and/or approved for human consumption.

Suitable stabilizers include sodium bicarbonate, potassium bicarbonate, sodium carbonate, potassium carbonate, calcium carbonate, magnesium carbonate, zinc carbonate, and any combination thereof. Other stabilizers include acids such as citric acid, phosphoric acid, adipic acid, ascorbic acid, lactic acid, malic acid, fumaric acid, gluconic acid, succinic acid, tartaric acid, maleic acid, and any combination thereof.

Other stabilizers include antioxidants such as, but are not limited to hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, vitamins and vitamin cofactors, such as vitamin A, vitamin C, vitamin E, vitamin E phosphate and ubiquinone (ubidecarenone, coenzyme Q, coenzyme Q10), ascorbic acid, citric acid, rosemary oil, minerals, such as mineral selenium and manganese, melatonin, alpha-carotene, .beta.-carotene, lycopene, lutein, zeanthin, crypoxanthin, resveratrol, eugenol, quercetin, catechin, gossypol, hesperetin, curcumin, ferulic acid, thymol, hydroxytyrosol, tumeric, thyme, olive oil, lipoic acid, glutathione, gulamine, oxalic acid, tocopherol-derived compounds, di-alpha-tocopheryl phosphate, tocotrienols, butylated hydroxyanisole, butylated hydroxytoluene, ethylenediaminetetraacetic acid, tert-butylhydroquinone, acetic acid, pectin, tocotrienol, tocopherol, coenzyme Q10 (coQ10), zeaxanthin, astaxanthin, canthaxanthin, saponins, limonoids, kaempferol, myricetin, isorhamnetin, proanthocyanidins, quercetin, rutin, luteolin, apigenin, tangeritin, hesperetin, naringenin, eriodictyol, flavan-3-ols (e.g., anthocyanadins), gallocatechins, epicatechin and its gallate forms, epigallocatechin and its gallate forms theaflavin and its gallate forms, thearubigins, isoflavone phytoestrogens, genistein, daidzein, glycitein, anythocyanins, cyaniding, delphinidin, malvidin, pelargonidin and peonidin. In one example, the antioxidant is vitamin C. In another example, the antioxidant is a coenzyme Q-containing compounds, such as ubiquinone (ubidecarenone, coenzyme Q, coenzyme Q10).

In some embodiments, the particulate compositions are suitable for direct ingestion by a human or other mammal. In some embodiments, the particulate compositions are suitable for dispersion and/or dilution in an aqueous solvent, e.g., water, juice, or other beverage. In embodiments, the clarity of the aqueous liquid upon dilution of the composition can be assessed by measuring the particle size and/or number of particles of the liquid. Methods for measuring particle size are known and any method for measuring particle size that can measure particle sizes in the appropriate ranges as described below, can be used.

The particulate compositions herein may be soluble in cold water, e.g., water at a temperature of about 20° C. or less. That is, the composition particles may dissolve in the water within 30 seconds, within a minute, or within a few minutes with gentle mixing to form a clear or translucent/somewhat cloudy solution, wherein the solution remains stable with minimal or no particles undissolved or settling out of solution for at least 5 minutes upon sitting without agitation. In some embodiments, the composition may be completely soluble in water at a temperature of 20° C. or greater, and at least partially soluble in water at a temperature less than 20° C., e.g., ranging from about 5° C. to 20° C. Further, for example, the composition may be completely soluble in water at a temperature of 10° C. or higher, and at least partially soluble in water having a temperature ranging from about 5° C. to 10° C. For example, the compositions herein may be characterized as having good, excellent, or fair solubility in water at a temperature ranging from 5° C. to 20° C., wherein a solubility time of less than 20 seconds=excellent solubility, 20-30 seconds=good solubility, 1-3 minutes=fair solubility, 3-5 minutes=poor solubility, and greater than 5 minutes=insoluble. Solubility of the particulate compositions can be measured by adding a 400 mg sample to 240 mL (8 oz.) of water at the specified temperature with continuous mixing at about 300 rpm. In an exemplary procedure, water added into 250 ml glass beaker set on a magnetic stirrer, and a magnetic stir bar (¾″ long) is added and set to about 300 rpm to create a slight vortex. A 400 mg sample of the test power is poured into the water, and the time for all particles to dissolve is measured. In some examples, the compositions herein may dissolve in 240 ml of water at a temperature less than or equal to 20° C. within 30 seconds, within 25 seconds, within 20 seconds, within 15 seconds, within 10 seconds, or within 5 seconds. The particulate compositions herein may be about the same or more soluble than sucrose (table sugar) under the same conditions.

Preferably, the average particle size of the discrete particles of the composition is less than or equal to about 1 micron, preferably less than or equal to about 500 nm, preferably less than or equal to about 100 nm, preferably less than or equal to about 50 nm, preferably less than or equal to about 10 nm, or less than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190 or 200 nm.

Likewise, in embodiments, when the particulate composition is subsequently diluted with an aqueous solvent, the average particle size, or more properly domain size of the cannabinoid in the resulting dispersion or solution is preferably, the average particle size of the particulate composition less than or equal to about 1 micron, preferably less than or equal to about 500 nm, preferably less than or equal to about 100 nm, preferably less than or equal to about 50 nm, preferably less than or equal to about 10 nm, or less than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190 or 200 nm.

Typically, it is desired that the aqueous liquid dilution compositions have a particle size less than or about less than 100 nm, less than or about less than 50 nm, or less than or about less than 25 nm. Typically, the particle size of the particulate composition or the mixture produced by dilution in an aqueous liquid is between or about between 5 nm and 200 nm, or between 5 nm or about 5 nm and 50 nm or about 50 nm.

Clarity of the liquid produced by the dilution of the particulate composition can be analyzed by taking optical turbidity measurements, which indicate the level of cloudiness or haziness of a liquid, correlating to the size and number of particles in suspension in a liquid. For example, turbidity can be measured optically, to get a value indicating the cloudiness or haziness of the liquid, which correlates with particles in suspension in the liquid. The units of a turbidity value measured with a nephelometer are expressed as Nephelometric Turbidity Units (NTU). The more clear a particular liquid, the lower its turbidity (i.e., NTU) value.

Turbidity can be measured optically, for example, using a nephelometer, an instrument with a light and a detector. The nephelometer measures turbidity by detecting scattered light resulting from exposure of the liquid to an incident light. The amount of scattered light correlates to the amount of particulate matter in the liquid. For example, a beam of light passes through a sample with low turbidity with little disturbance. Other methods for measuring turbidity are well known and can be used with the provided methods and compositions.

The mixture produced by dilution of embodiments of the particulate composition forms a nanoemulsion having a low turbidity, for example, a turbidity value (NTU) less than or about 80, such as less than or about 70, less than or about 60, less than or about 50, less than or about 40, less than or about 30, less than or about 29, less than or about 28, less than or about 27, less than or about 26, less than or about 25, less than or about 24, less than or about 23, less than or about 22, less than or about 21, less than or about 20, less than or about 19, less than or about 18, less than or about 17, less than or about 16, less than or about 15, less than or about 14, less than or about 13, less than or about 12, less than or about 11, less than or about 10, less than or about 9, less than or about 8, less than or about 7, less than or about 6, less than or about 5, less than or about 4, less than or about 3, less than or about 2, less than or about 1, or about 0. For example, the turbidity value of the aqueous liquid dilution compositions provided herein typically is less than or about 80, for example, 80, 70, 60, 50, 40, 30, 25, 20, 15, 10, 5, 4, 3, 2, 1 or less. The turbidity depends upon the components of the compositions and amounts thereof.

### Stability

In embodiments, the particulate composition or the liquids produced by dilution of the particulate composition are free from one or more changes over a period of time, for example, 1 or more days, 1 or more weeks, 1 or more months, or one or more years, for example, 1, 2, 3, 4, 5, 6, 7 or more days, 1, 2, 3, 4, 5, 6, 7, 8, 9, 10 or more weeks, 1, 2, 3, 4, 5, 6, 7, 8, 9, 10, 11, 12 or more months or 1, 2, 3, 4 or more years.

### Oral Bioavailability

In one or more embodiments, the particulate composition or the liquids produced by dilution of the particulate composition exhibit a high or relatively high bioavailability, for example, a bioavailability that is higher than a liquid containing the cannabinoid alone (i.e., not formulated according to embodiments disclosed herein). Bioavailability relates to the ability of the body to absorb the cannabinoid into a particular space, tissue cell and/or cellular compartment. Typically, cannabinoids in nano emulsions according to embodiments disclosed herein are better absorbed than those with larger particle sizes.

In embodiments, the particles or nanofibers of the composition are disposed on a substrate. Suitable substrates include any solid or semisolid material capable of supporting a plurality of discrete particles of the particulate compositions according to one or more embodiments disclosed herein.

Suitable substrates include films, wafers or sheets, and the like which can be formed to include a mixture comprising the particulates comprising the one or more cannabinoids and the polymeric carrier. Films generally have an aspect ratio of thickness to width along any length of greater than or equal to 100. Films are generally considered to have a thickness of less than or equal to about 1 mm. Suitable substrates include films which are sublingual or orally dissolving, e.g., mucosally dissolving films, generally considered to be edible and pharmaceutically acceptable. A “mucosally dissolvable film” refers to any film that allows an active agent to be released into mucosal fluid and/or absorbed through one or more mucosal membranes of any mammalian subject.

Other types of films suitable for use herein include oral films which may be swallowed and/or which dissolve or otherwise disperse when contacted with mucosal fluid and/or an aqueous liquid, allowing the biologically active component, i.e., the cannabinoid, to traverses the digestive tract of the subject.

In one or more alternative embodiments, the substrate may adhere to any mucosal tissue of a subject and/or may adhere to an epidermal portion of an intended used allowing for and facilitating transdermal transport of the cannabinoid into the metabolic pathways of the end user.

In one or more embodiments, the substrate comprises and/or forms an emulsion comprising the cannabinoid when dissolved and/or dispersed in mucosal fluid and/or another bodily fluid.

In addition to the cannabinoids and/or other biologically active agents or components, the substrate according to embodiments of the instant disclosure may also comprise permeability and/or penetration enhancers and/or absorption enhancers to improve the absorption of the active agent by the mucosal tissues or other metabolic systems of a subject. Other components may include taste-masking agents or bitter blockers to mask the bitter taste of cannabinoids.

Suitable penetration enhancers include calcium chelators such as EDTA, polycarboxylic acids, zonula occluding toxin, poly-L-arginine, chitosan derivatives, niacin, omega 3 or 6 fatty acids or other fatty acids, menthol, sodium caprate, sodium deoxycholate, dipotassium glycyrrhizinate, 25 furanocoumarins and grapefruit derivatives, bile salts, ethylenediaminetetraacetic acid, and the like.

In embodiments, penetration and/or absorption enhancers may be present in the substrate from about 0.001 wt % to about 10 wt %, based on the total weight of the substrate present.

In one or more embodiments, suitable taste-masking agents include kleptose, cyclodextrin, cyclodextrin derivatives, ginger, anise, cinnamon, peppermint, licorice, fruit flavoring, citric acid, fruit juice, sweeteners, sucrose, glucose, fructose, mannitol, saccharin, aspartame, sucralose, Stevia plant derivatives, honey, derivatives thereof, and combinations thereof. In embodiments, one or more taste-masking agents are present in the substrates from about 0.001 wt % to about 5 wt %, based on the total amount of the substrate present.

In one or more embodiments, the substrate may further include one or more of a film-forming agent; a filler; a plasticizer; a taste-masking agent; a coloring agent; a solubilizing agent; an effervescent agent; an antioxidant; an absorption enhancer; a disintegrating agent; a pH modifying or buffering agent; a surfactant; a complexing agent; a bio-adhesive agent; a sheet adhesive; an identifying agent; an anti-counterfeiting agent; a tracking agent; transporter inhibitor agent; transporter inducer agent; emulsifying agent, self-emulsifying system agents; crystallization inhibitor; crystallization promoter; super-saturation promoting agent; antimicrobial preservative; catalyst; chelating agent; particles; organoleptic agent; flavoring agent; scent agent; identifying device; and/or anti-counterfeiting device.

In one or more embodiments, the substrate comprises one or more cellulosic materials, preferably selected from methylcellulose, hydroxypropylmethylcellulose, ethylcellulose, sodium alginate, poly (methacrylic acid-co-ethyl acrylate), poly (methacrylic acid-co-methyl methacrylate), starch, 30 polyvinylpyrrolidone, polylactic acid (PLA), poly-L-lactide (PLLA), poly-D-lactide (PLDA), poly (lactic-co-glycolic acid) (PLGA), chitosan, chitin, pullulan, derivatives thereof, and combinations thereof. The plasticizer when used in the preparation of the substrate may be selected from glycerine, triacetin, triacetyl citrate, polyethyleneglycol, mineral oil, myglyol, derivatives thereof, and combinations thereof.

Suitable effervescent agents include sodium bicarbonate, potassium bicarbonate, citric acid, malic acid, tartaric acid, adipic acid, fumaric acid, derivatives thereof, and combinations thereof.

In one or more embodiments, the substrate further includes one of more antioxidants, which may include tocopherol, a tocopheryl polyalkylene glycol derivative, e.g., a tocopheryl polyalkylene glycol derivative, resveratrol, ascorbyl palmitate, tert-butylhydroquinone, resveratrol, nordihydroguaiaretic acid, cysteine, propyl gallate, octyl gallate, 3-tert-butyl-4-hydroxyanisole, butylated hydroxytoluene, ascorbic acid, derivatives thereof, and combinations thereof, and/or the like.

In embodiments, the substrate may include a disintegrating agent, preferably selected from croscarmellose sodium, sodium starch glycolate, insoluble polyvinylpyrrolidone, carboxymethylcellulose, derivatives thereof, and combinations thereof, and/or the like.

In one or more embodiments, the substrate may also include a pH modifier or pH buffer agent comprising one or more of sodium carbonate, magnesium carbonate, calcium carbonate, sodium hydroxide, potassium hydroxide, ascorbic acid, citric acid, succinic acid, fumaric acid, derivatives thereof, and combinations thereof.

In embodiments, the substrate further includes one or more surfactants and/or surfactant pairs. Suitable surfactants include sodium lauryl sulfate, fatty acid ethoxylates, EO-PO block copolymers, poloxamers, sorbitan esters, polysorbates, sorbitans, stearic acid, polyethylene glycols, derivatives thereof, and combinations thereof.

In embodiments, the substrate may include a complexing agent comprising cyclodextrins, calcium glycerophosphate, dodecyl 2-(N,N-dimethylamino) propionate, zinc, dextran, pectin, copper acetate, sodium deoxycholate, calcium, magnesium, derivatives thereof, and combinations thereof.

In one or more embodiments, the substrate may further include gelatin, starch, glycoproteins, proteins, carbohydrates, mucopolysaccharides, derivatives thereof, and combinations thereof, and/or one or more of polyvinylpyrrolidone, polyvinyl alcohol, polyvinyl acetate, confectionary glue, starch, derivatives thereof, or combinations thereof.

Suitable tracking agents, identifying agents, or anti-counterfeiting agents, and the like may include particular combinations of fluorescein, rhodamine, succinimidyl esters, maleimide activated fluorophores, fluorescent dyes, fluorescent particles, infrared active particles, near infrared active particles, metallic nanoparticles, polymeric particles, silica based nanoparticles, SERS (Surface Enhanced Raman Spectroscopy) particles, raman active particles, derivatives thereof, and combinations thereof.

In embodiments, the substrate may further include an osmotic agent, preferably selected from mannitol, osmitrol, dextrose, sucrose, fructose, sodium chloride, potassium chloride, xylitol, sorbitol, lactose, potassium phosphate, derivatives thereof, or combinations thereof.

In one or more embodiments, the substrate may include a transporter inhibitor. Suitable examples include elacridar, zosuquidar, glibenclamide, quinaxoline derivatives, phenylalanine, arginyl naphthylamide, grapefruit derivatives, furanocoumarins, derivatives thereof, and/or one or more transporter inducers such as xenobiotics, diallyl sulfide, dexamethasone, derivatives thereof, and combinations thereof.

In addition, the substrate may further include non-ionic polyethoxylates, polyethylene glycols, polyethylene-polypropylene glycols, cholesterols, octyldodecanol, polyoxylglycerides, derivatives thereof, and combinations thereof.

Suitable self-emulsifying systems include Labrasol, Labrafil, Cremophor, Pluronics, Lutrol, poloxamers, polysorbates, ethyl linoleate, mono- and diglycerides of capric and caprylic acids, tocopherol acetate, Solutol, soybean oil, and the like.

In one or more embodiments the substrate may include a crystallization inhibitor such as polyvinylpyrollidone, hydroxypropylmethylcellulose, silicon dioxide, dextrins, dextrans, bile acids, sterols, polysebacic anhydride, derivatives thereof, and combinations thereof.

Other suitable components present in the substrate include supersaturating promoting agents such as hydroxyproylmethylcellulose, hydroxypropylmethylcellulose acetate succinate, polyvinylpyrollidone, derivatives thereof, and combinations thereof.

In some embodiments, the substrate may include an antimicrobial agent, and/or a preservative such as, for examples, benzoic acid, sodium benzoate, methyl paraben, propyl baraben, butyl paraben, sorbic acid, propionic acid, dehydroacetic acid, derivatives thereof, and combinations thereof.

In one or more embodiments, the substrate may further include an organoleptic agent such as a flavorant or scent. Suitable components include those associated with vanilla, bubble gum, fruit flavor, mint, chocolate, licorice (anise), marshmallow, peanut butter, aspartame, sucralose, sucrose, glucose, citric acid, stevia plant, derivatives thereof, or combinations thereof. In alterative embodiments the organoleptic agent may include glutamates, chicken flavor, umami flavoring, beef flavor, fish flavor, or the like. Suitable chelating agents for use herein include disodium edetate, EDTA, pentetic acid, derivatives thereof and combinations thereof.

In one or more embodiments the substrate includes starches such as corn starch, potato starch, pregelatinized and modified starches thereof, cellulosic agents such as Act-di-sol, montmorrilonite clays including cross-linked PVP, sweeteners, bentonite, gums, microcrystalline cellulose, alginates, sodium starch glycolate, gums such as agar, guar, locust bean, karaya, pecitin and/or tragacanth. Suitable disintegrants and/or glidants include silica.

In may comprise up to about 20 weight percent and preferably between about 2 and about 5 percent of the total weight of the composition.

In addition to cannabanoids, the substrate may further include other biologically active components. Suitable examples include vitamins and/or other trace organic and/or inorganic substances required by a particular diet. Examples include thiamin, riboflavin, nicotinic acid, pantothenic acid, pyridoxine, biotin, folic acid, vitamin B12, lipoic acid, ascorbic acid, vitamin A, vitamin D, tocopheryl polyalkylene glycol and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), nicotinamide adenine dinucleotide (AND), nicotinamide adenine dinucleotide phosphate (NADP), Coenzyme A (CoA), pyridoxal phosphate, biocytin, tetrahydrofolic acid, coenzyme B12, lipoyllysine, 11-cis-retinal, and 1,25-dihydroxycholecalciferol. The term vitamin(s) also includes choline, carnitine, and alpha, beta, and gamma carotenes, and/or minerals such as calcium, iron, zinc, selenium, copper, iodine, magnesium, phosphorus, chromium and the like, and mixtures thereof.

In embodiments, the substrate may include a dietary supplement such as, for example bee pollen, bran, wheat germ, kelp, cod liver oil, ginseng, fish oils, amino-acids, proteins and mixtures thereof.

In one or more embodiments the substrate may include binders such as but not limited to acacia, tragacanth, gelatin, starch, cellulose materials such as methyl cellulose and sodium carboxymethyl cellulose, alginic acids and salts thereof, polyethylene glycol, guar gum, polysaccharide, sugars, invert sugars, poloxomers, collagen, albumin, gelatin, cellulosics in nonaqueous solvents, and combinations of the above and the like. Other binders include, for example, polypropylene glycol, polyoxyethylene-polypropylene copolymer, polyethylene ester, polyethylene sorbitan ester, polyethylene oxide or combinations thereof and the like. Binders may be hydrophilic or hydrophobic.

In embodiments, the substrate is in the form of a film. As use herein, a film refers to a thin sheet-like material having a plurality of sides and forming essentially any shape, e.g., rectangular, square, circular, or the like. The films described herein may be any desired thickness and size suitable for the intended use. Suitable examples include those sized such that it may be placed into the oral cavity of the user. Other films may be sized for application to the skin of the user, i.e., a topical use. For example, some films may have a relatively thin thickness of from about 0.1 to about 10 mils, while others may have a somewhat thicker thickness of from about 10 to about 30 mils. For some films, especially those intended for topical use, the thickness may be even larger, i.e., greater than about 30 mils. In addition, the term “film” includes single-layer compositions as well as multi-layer compositions, such as laminated films, coatings on films and the like. The composition in its dried film form maintains a uniform distribution 5 of components through the application of controlled drying of the film.

In embodiments, the substrate is a film having a substantially uniform thickness. In alternative embodiments, the thickness of the film is anisotropic.

In one or more embodiments, the substrate comprises a plurality of layers. In some embodiments, the thickness of each layer may be from about 100 nm to about 500 microns. Nano-scale sheets can range from about 100 nm to about 1000 nm, from about 200 nm to about 900 nm, from about 300 nm to about 800 nm, from about 400 nm to about 700 nm, or from about 500 to about 600 nm. The micron-scale sheets can range from about 1 micron to about 1000 microns, from about 10 microns to about 250 microns, from about 20 microns to about 200 microns, from about 30 microns to about 150 microns, from about 10 40 microns to about 125 microns, from about 50 microns to about 100 microns, from about 60 microns to about 90 microns, or from about 70 microns to about 80 microns. However, it should be recognized that the sheets can have any thickness that allows for preparation into an ingestible unit as described herein. In one example, each discrete sheet has a thickness less than 50 microns.

In embodiments, the substrate comprises a plurality of discrete layers, wherein some of the layers are different in composition compared to other layers. For example, one or more layers may include various components listed herein, while others include the particulates comprising the one or more cannabinoids at least partially encapsulated by the polymeric carrier according to embodiments disclosed herein, each having a different dissolution rate to allow for a controlled release of the various cannabinoids into the end user's metabolic systems.

The substrate may comprise a laminate which is formed via coextrusion, and/or the various layers of the laminate may be combined in one or more processes.

In embodiments, the cannabinoids are present within the substrate at from about 0.1 wt % to about 50 wt %, based on the total amount of the substrate present. The cannabinoids may be purified, e.g., having at least about 90% purity, and/or the cannabinoids may be an extract, wax, and/or oil isolated from hemp and/or another form of cannabis.

In one or more embodiments, the cannabinoid present within the substrate comprises cannabidiol, a tetrahydrocannabinol, a derivative thereof, or a combination thereof. In other embodiments, the cannabinoid present within the substrate consists essentially of cannabidiol and/or a derivative thereof.

### Embodiments Listing

Having described the various embodiments of the disclosure herein, further specific embodiments include those set forth in the following paragraphs:
E1. A composition comprising a plurality of discrete particles comprising one or more cannabinoids disposed at least partially within a polymeric carrier having a maximum overall dimension of less than 1 micron.
E2. The composition of embodiment E1, produced by electrospray of a solution comprising one or more cannabinoids and the polymeric carrier.
E3. The composition of embodiment E1 or E2, produced by coaxial electrospray including an outer flow comprising the polymeric carrier, and an inner flow comprising the one or more cannabinoids.
E4. The composition of any one of embodiments E1 through E3, wherein the one or more cannabinoids include a tetrahydrocannabinol (THC), a cannabidiol (CBD), a cannabivarin (CBV), a tetrahydrocannabivarin (THCV), or a combination thereof.
E5. The composition of any one of embodiments E1 through E4, wherein the polymeric carrier includes a gelatin, ethyl cellulose, or a combination thereof.
E6. The composition of any one of embodiments E1 through E5, comprising greater than or equal to about 30 wt % of the one or more cannabinoids.
E7. The composition of any one of embodiments E1 through E6, wherein a 10 wt % mixture of the composition in water at 25° C. forms a clear solution.
E8. A composition comprising a plurality of discrete nanofibers comprising one or more cannabinoids disposed at least partially within a polymeric carrier having a maximum width of less than 1 micron.
E9. The composition of embodiment E8, produced by electrospinning of a solution comprising one or more cannabinoids and the polymeric carrier.
E10. The composition of embodiment E8 or E9, produced by coaxial electrospinning including an outer flow comprising the polymeric carrier, and an inner flow comprising the one or more cannabinoids.
E11. The composition of any one of embodiments E8 through E10, wherein the one or more cannabinoids include a tetrahydrocannabinol (THC), a cannabidiol (CBD), a cannabivarin (CBV), a tetrahydrocannabivarin (THCV), or a combination thereof.
E12. The composition of any one of embodiments E8 through E11, wherein the polymeric carrier includes a gelatin, ethyl cellulose, or a combination thereof.
E13. The composition of any one of embodiments E8 through E12, comprising greater than or equal to about 30 wt % of the one or more cannabinoids.
E14. The composition of any one of embodiments E8 through E13, wherein a 10 wt % mixture of the composition in water at 25° C. forms a clear solution.
E15. A process to produce a composition according to any one of embodiments E1 through E7, comprising the steps of:
   a) providing one or more precursor mixtures comprising one or more cannabinoids and one or more polymeric carrier components in a solvent; and
   b) electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles.
E16. A process to produce a composition according to any one of embodiments E8 through E14, comprising the steps of:
   a) providing one or more precursor mixtures comprising one or more cannabinoids and one or more polymeric carrier components in a solvent; and
   b) electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.
E17. A process to produce a composition comprising the steps of:
   a) providing one or more precursor mixtures comprising one or more cannabinoids and one or more polymeric carrier components in a solvent; and
   i) electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles; or
   ii) electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.
E18. A process to produce a composition comprising the steps of:
   a) providing one or more precursor mixtures comprising one or more cannabinoids and one or more polymeric carrier components in a solvent; and
   b) electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles.
E19. A process to produce a composition comprising the steps of:
   a) providing one or more precursor mixtures comprising one or more cannabinoids and one or more polymeric carrier components in a solvent; and
   b) electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.
E20. The process according to any one of embodiments E15 through E19, wherein a first precursor mixture comprises one or more cannabinoids in a solvent;
   a second precursor mixture comprises one or more polymeric carrier components dissolved and/or dispersed in a solvent; and
   each of the precursor mixtures are coaxially electrosprayed to form the composition.
E21. The process according to any one of embodiments E15 through E20, wherein the one or more cannabinoids include a tetrahydrocannabinol (THC), a cannabidiol (CBD), a cannabivarin (CBV), a tetrahydrocannabivarin (THCV), or a combination thereof.
E22. The process according to any one of embodiments E15 through E21, wherein the composition comprises greater than or equal to about 30 wt % of the one or more cannabinoids.

### Examples

The foregoing discussion can be further described with reference to the following non-limiting examples.

For purposes herein, the following abbreviations are used:

| | |
|---|---|
| AA | Acetic acid |
| CBD | Cannabidiol |
| DCM | Dichloromethane |
| DMAc | Dimethylacetamide |
| DMF | Dimethylformamide |
| ECU | Environmental control unit |
| FDA | Food and Drug Administration |
| HA | Hyaluronic acid |
| HFP | 1,1,1,3,3,3-Hexafluoro-2-propanol |
| MeOH | Methanol |
| Mn | Number average molecular weight |
| Mw | Number average molecular number |
| PCL50 | Polycaprolactone Mw-50,000 |
| PCL80 | Polycaprolactone Mn-80,000 |
| SEM | Scanning electron microscope |
| THC | Tetrahydrocannabinol |
| VC | Vitamin C |
| VE-TPGS | Vitamin E-TPGS |

The following examples and experiments confirm the ability to incorporate CBD and THC into unique nanoparticles and nano fibers through the electrospraying and electrospinning techniques using commercially available equipment, (Bioinicia Fluidnatek LE-100; Bioinicia, Spain) equipped with an atmosphere control unit (Thermo Scientific, USA) under tight temperature and relative humidity conditions. The examples demonstrate nanoscale particles containing CBD in single-phase to support slow release, nanoscale particles containing CBD in single-phase to support fast release, nanoscale particles containing THC in single-phase, and CBD containing scaffold able to quickly dissolve. Polymeric scaffold containing vitamin C and hyaluronic acid able to retain its integrity was also shown.

Sample solutions were prepared according to the following Table 1.

TABLE 1

| | SAMPLE |
|---|---|
| DESCRIPTION | NUMBER |
| 6 WT % GELATIN IN HFP WITH 25 MG CBD PER ML | 1 |
| HFP | |
| 12 WT % PCL80:VC:HA (79.9:20:0.1 W/W) IN | 2 |
| DCM:DMF:H2O (69:30:1 W/W) | |
| 1.5 WT % PCL50 IN CHLOROFORM:MEOH (1:1 W/W) | 3 |
| 1.5 W/V % PCL50 IN AA | 4 |
| 1.5 W/V % PCL50 IN AA | 5 |
| 1.5 W/V % PCL50:CBD (90:10 W/W) IN AA(AQ) + | 6 |
| 0.002% V/V TWEEN-20 | |
| 3 W/V % PCL:CBD:VE-TPGS (86:10:4 W/W) IN AA | 7 |
| 1.5 W/V % PCL50:THC:TWEEN-20 IN AA | 8 |
| 1.5 W/V % PCL50:THC:TWEEN-20 IN AA | 9 |
| 1.5 W/V % PCL50:THC:TWEEN-20 IN AA | 10 |
| 1.5 W/V % PCL50:THC:TWEEN-20 IN AA | 11 |
| 8 W/V % GELATIN IN 20% V/V AA | 12 |
| 2 WT % GELATIN IN HFP + 10 MG CBD PER ML HFP | 13 |
| 2 WT % GELATIN IN HFP + 10 MG CBD PER ML HFP | 14 |
| 8 W/V % GELATIN IN 50% V/V AA(AQ) + 1 MG CBD | 15 |
| PER ML | |

Electrospun scaffolds were deposited on top of a rotating drum with a substrate made of polyethylene film. Electrosprayed particles were deposited on a flat plate collector with a substrate made of polyethylene film.

### CBD Containing Scaffold Able to Quickly Dissolve

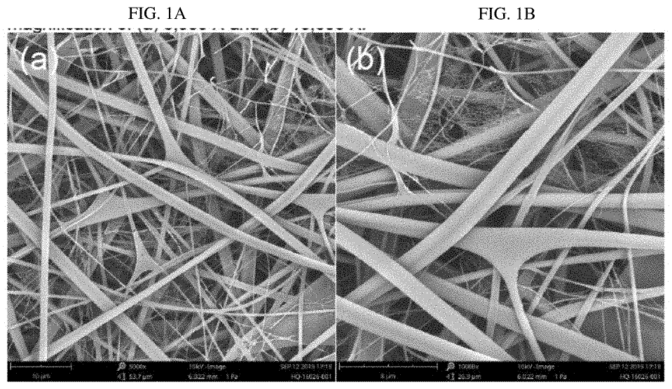

FIG. 1 shows the microstructure of gelatin electrospun fibers produced using Sample 1, containing CBD isolate at two different magnifications. Typical ribbon like fibers are seen, and even smaller web-like fibers are present. A quick exposure to deionized water completely dissolved the electrospun strip created, which may support instantaneous cannabinoid delivery.

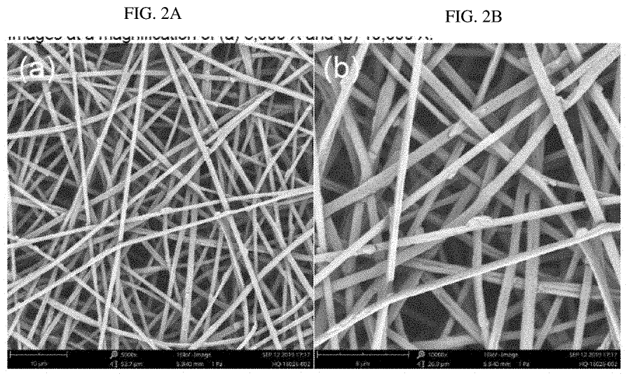

Next, Sample 2 was electrospun pursuant to demonstration of slow delivery of a cannabinoid via electrospun scaffolds that do not dissolve quickly in the presence of water. FIG. 2 shows the microstructure of PCL80 containing VC and HA on its structure. Once exposed to water, VC was released from the electrospun fiber, followed by HA. The microstructure also shows homogeneous distribution of particulate, which are composed of blended VC and HA. This uniform distribution is due to a homogeneous and well mixed solution prior to sample development which resulted in a uniform distribution throughout the fiber structure.

Similar to the gelatin electrospun scaffold, optimization of the sample properties was tailored to faster degradation by incorporating fast degrading polymers, slow or fast release of the cannabinoid by controlling fiber diameter, adding porogens, among others.

### Nanoscale Particles Containing THC in Single-Phase

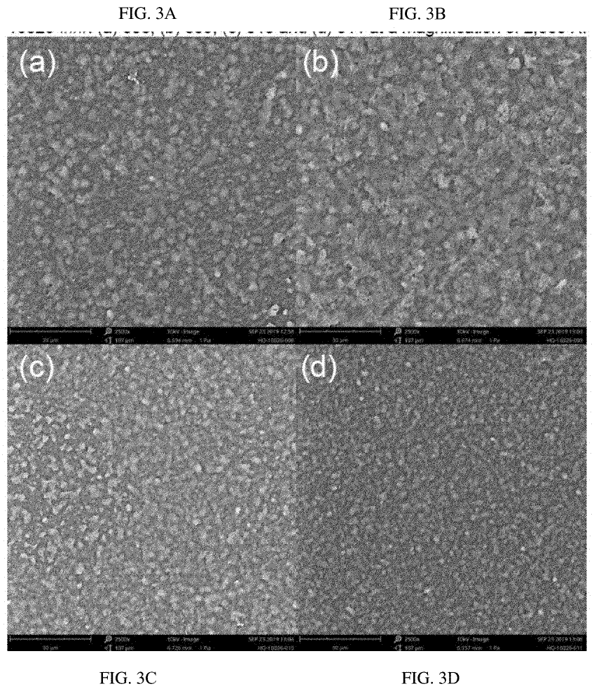

THC was selected to prepare an electrosprayed solution using Tween-20 as the surfactant to aid in particle suspension. FIG. 3 shows multiple microstructures (a), (b), (c), and (d) of Samples 8, 9, 10, and 11 respectively at 2,500×, where variations of electrosprayed parameters were studied. While a particle-like structure is seen, rounded particles with well-defined structure were not seen.

### Nanoscale Particles Containing CBD in Single-Phase for Slow Release

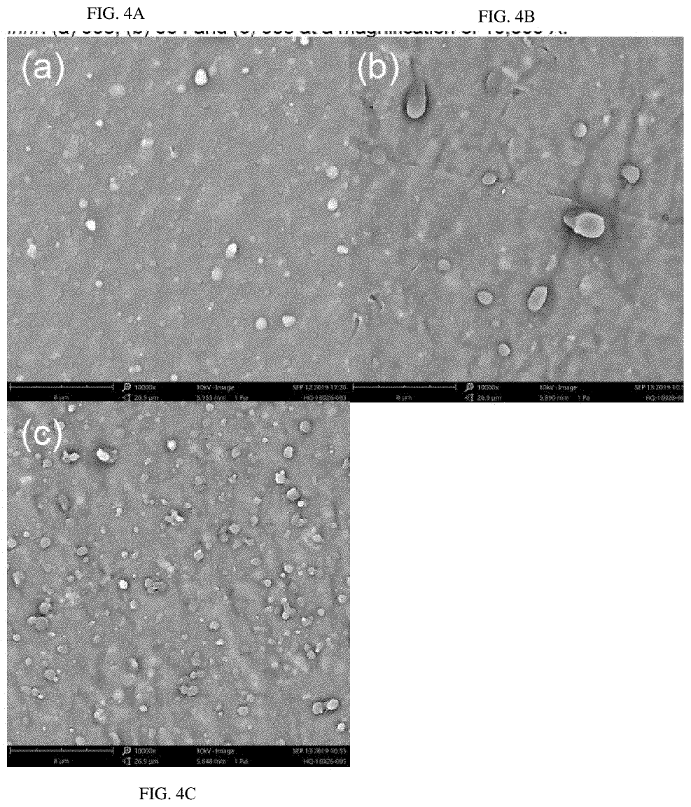

In order to achieve nanoscale sized particles, methanol was incorporated in the electrospraying process due to its higher dielectric constant relative to polymers currently under evaluation. It was discovered that increasing the dielectric constant in solution, the process is forced to decrease the diameter of the particle due to an increase in conductivity. Nanoscale particles with PCL50 and Tween-20 were obtained by electrospraying Samples 3, 4, and 5 and are shown in in FIGS. 4 (a), (b), and (c) respectively at a magnification of 10,000×.

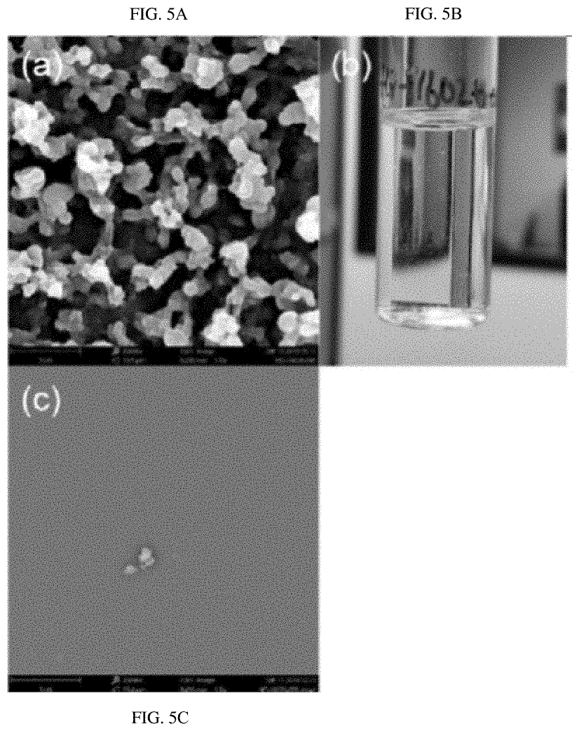

To study how these particles behaved in water, Sample 6 was electrosprayed for 3 hours to obtain enough material for a trial. FIG. 5a shows the particle morphology after sample development. FIG. 5b shows the particles exposed to 10 mL of deionized water and after agitation. Agglomerated particles are seen suspended in the water. This solution was left resting until all agglomerated particles fell at the bottom of the vial due to gravity. To proof that nanoscale particles were still suspended in the apparent clear top side, a small aliquot was taken, and water left to dry. This area was then imaged in the SEM and single particles were found (FIG. 5c), proving that some particulate remains suspended. These results confirm agglomeration can be fostered and/or avoided, and well dispersed particles can be obtained.

CBD contained in PCL50 and VE-TPGS particles were created following the same electrosprayed procedure as for Sample 6, were methanol, not in solution, but during the electrospraying process was used to create nanoparticles as shown in FIGS. 6 (a) and (b) at 10,000× and 20,000× respectively.

Similar to Sample 6, dry CBD containing nanoscale particles were exposed in water to see their capability to remain suspended over time. While agglomerates form, observed in solution Sample 7 which settled at the bottom of the vial due to gravity and particle weight, individual particles remained in the supernatant of the vial.

Accordingly, these examples suggest that the use of alternate surfactants provides a solution to obtain particles that can remain suspended over long periods of time while exposed to water-based solutions. In addition, these samples suggest that the selection of the polymeric carrier affects the dissolution of the particles.

### Nanoscale Particles Containing CBD in Single-Phase for Fast Release

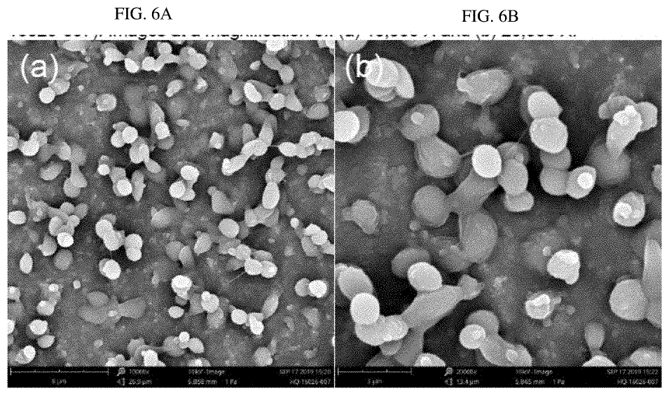

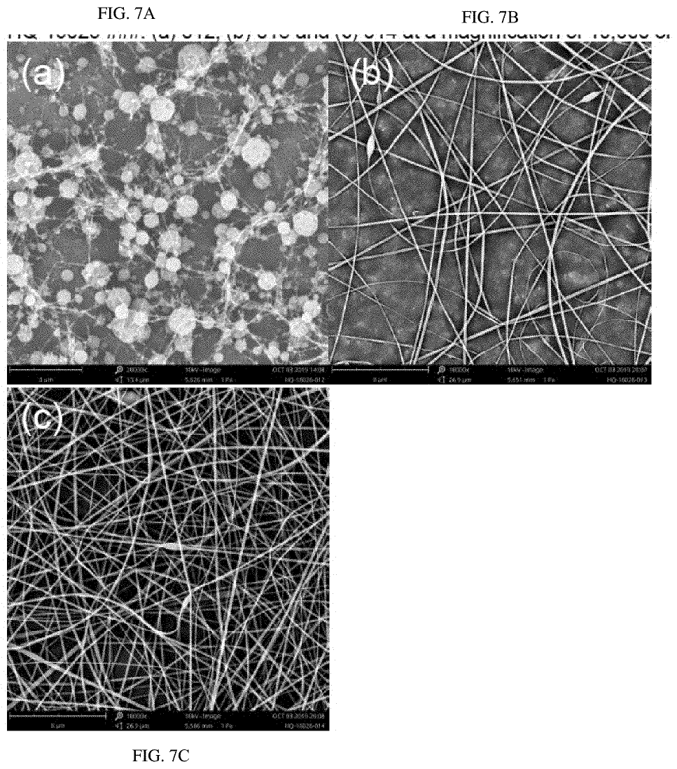

Fast dissolving particles were produced using edible gelatin nanoscale particles created from acetic acid-water based solutions. A solution was created, and the electrospray process was repeated to see if reproducible results were obtainable. FIG. 7a shows nanoscale gelatin-based particles produced from Sample 12 at 10,000×. Since CBD is not able to dissolve in water, a similar solution was prepared but with HFIP in which CBD is soluble. Samples 13 (FIG. 7(b), and Sample 14 (FIG. 7(c) possessed relatively low viscosity, which resulted in less than optimal fibers being obtained as shown in FIG. 7(b) and FIG. 7(c) at 20,000×.

In order to create CBD particles in a gelatin-based solution, the solubility of CBD in different acetic acid-water based solutions was explored. Emulsions were formed for 30 and 40% v/v AA(aq.), 50% v/v AA(aq.) and higher concentrations demonstrated a clear, homogeneous and well mixed soluble CBD solution. Based on these results, and since higher water content is typically needed to create nanoscale particles, a gelatin solution created with the 50% v/v AA and CBD was created (Sample 15).

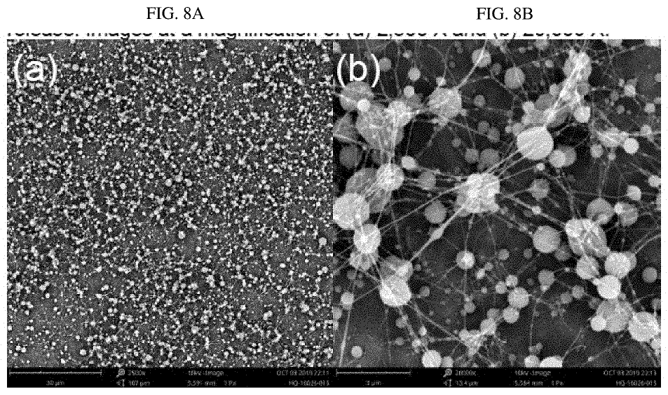

FIG. 8 (a) shows the microstructure obtained at 2,500× and FIG. 8(b) shows the same at 20,000×, where nanoscale particles are seen. Although a small presence of fibers throughout the sample are seen, these results confirm the formation of nanoparticles according to embodiments of the instant disclosure.

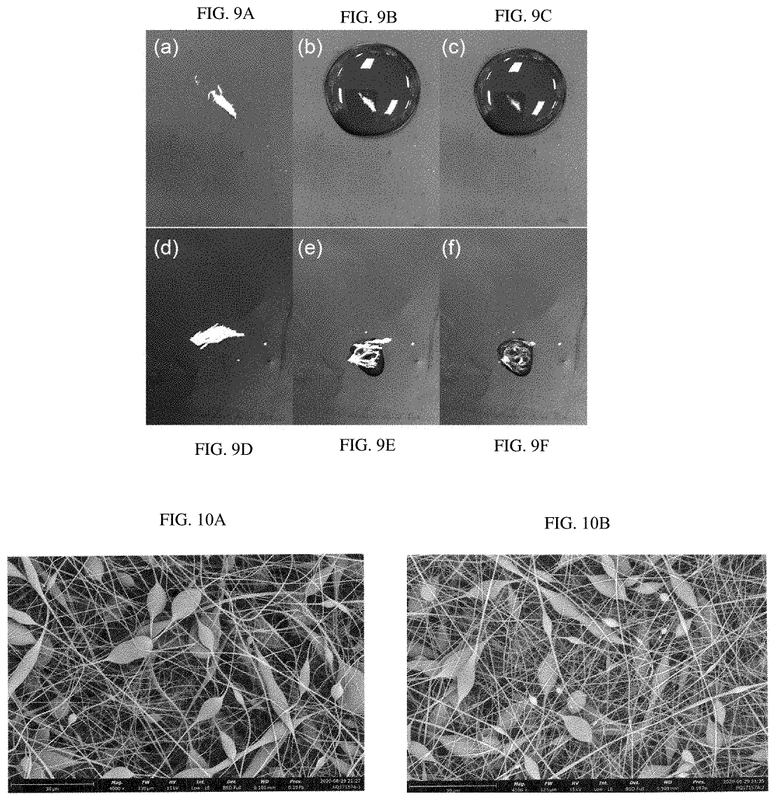

It was discovered that the presence of these smaller fibers in the particles obtained by electrospraying of Sample 15 appear to hold together the particles giving them a weak tactile feeling of a dry laboratory wipe. Once the particles were exposed to water, the particles quickly dissolved over a period of up to two minutes. FIG. 9 (a) through (c) and FIG. 9 (d) through (f) show two different samples of the particles which were than exposed to water over a period of 5 minutes. As these figures show, both instances the particles dissolved over time.

### Electrospinning of Ethyl Cellulose Nanofibers Including THCV

Several examples of electrospinning were conducted to produce compositions comprising a plurality of nanofibers in which the cannabinoid THCV was encapsulated within the polymeric carrier. In these examples, a solution containing about 10 wt % ethyl cellulose and about 20 wt % THCV in ethanol was directed through a five needle multi-emitter electrospinning system (Bioinicia Fluidnatek LE-100, Valencia Spain) under various conditions. Electrospinning produced nanofibers which were electrospun onto a flat plate with a carbon black infused polyethylene as the substrate. Typical conditions for these samples included a flowrate of between 0.5 and 1.5 ml/min, a distance between the spray needle and the collector from about of about 5 cm to about 10 cm, a needle voltage from about 10 kV to about 15 kV with the collector at −1V, ambient temperature of about 23° C., a relative humidity in the spray chamber maintained between about 35% and 45%, and a sheath gas airflow on the order of 60 to about 80 m³/hr. Samples were obtained in 5 minute intervals.

The SEM analysis of the compositions produced in two of these experimental runs are shown in FIGS. 10a and 10b in which 6.7 g and about 10 g of the electrospun fibers were produced, respectively. As these figures show, the nanofibers include a beaded structure with an average width or diameter (i.e., the dimension taken perpendicular to the length) from about 200 to about 400 nm. The fibers had around 6000 times more surface area than would be available if present in a spherical form. Both of the examples shown in the FIGS. 10a and 10b had a THCV content of over 70 wt %, and both were readily soluble in water, i.e., 10 10 wt % mixture of the composition in water formed a clear solution at 25° C.

A series of experiments was conducted utilizing a 20 wt % solution of THCV in ethanol. The polymeric carriers included gelatin—(175 bloom, Type A from porcine skin (Electron Microscopy Sciences), and ethyl cellulose (100 cP @ 5% in toluene:EtOH 80:20, 48% ethoxyl, Sigma-Aldrich). The compositions were produced using a five needle multi-emitter electrospinning system (Bioinicia Fluidnatek LE-100, Valencia Spain) under various conditions.

The experiments produced a plurality of nanofibers in which the THCV was encapsulated within the polymeric carrier. SEM micrographs of two examples of the electrospun nanofibers are shown in FIGS. 11a and 11b. The compositions produced had a THCV concentration from about 65 wt % to 70 wt %, and were readily soluble in water.

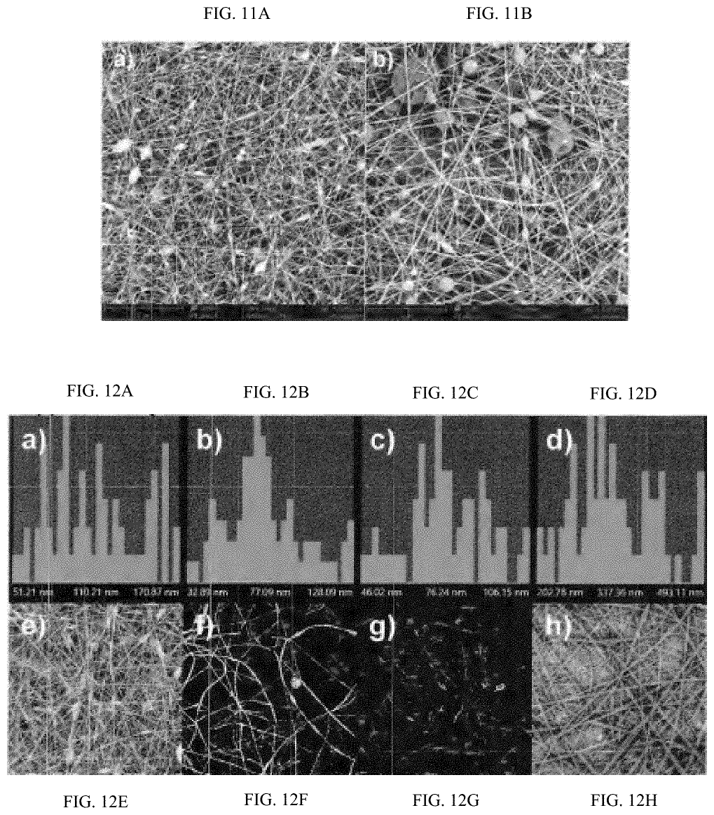

FIG. 12a through 12d shows a fibermatic analysis of the compositions shown in FIG. 12e through 12f, respectively. As these data show, the average width of the fibers is from about 75 nm to about 350 nm.

These examples demonstrated the feasibility to incorporate various cannabinoids into both electrosprayed particles and electrospun fibers. The particle diameter of CBD containing particles was sub-micron in scale and agglomerates can be produced. Both may be tailored for slow release or fast release depending on the polymeric carriers employed. These results provide proof that fast and/or slow release can be achieved with electrosprayed particles, electrospun fibers, or by creating a sample with both techniques simultaneously.

The following description is made for the purpose of illustrating the general principles of the present disclosure and is not meant to limit the inventive concepts claimed herein. Further, particular features described herein can be used in combination with other described features in each of the various possible combinations and permutations.

The above description is made for the purpose of illustrating the general principles of the present disclosure and is not meant to limit the inventive concepts claimed herein. Further, particular features described herein can be used in combination with other described features in each of the various possible combinations and permutations.

It should be noted that in the development of any such actual aspect, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure. In addition, the device, system and/or method used/disclosed herein can also comprise some components other than those cited.

Unless otherwise specifically defined herein, all terms are to be given their broadest possible interpretation including meanings implied from the specification as well as meanings understood by those skilled in the art and/or as defined in dictionaries, treatises, and the like.

It must also be noted that, as used in the specification and the appended claims, the singular forms “a,” “an” and “the” include plural referents unless otherwise specified.

As also used herein, the term “about” denotes an interval of accuracy that ensures the technical effect of the feature in question. In various approaches, the term “about” when combined with a value, refers to plus and minus 10% of the reference value. For example, a thickness of about 10 angstroms (Å) refers to a thickness of 10 Å+/−1 Å, e.g., from 0.9 Å to 1.1 Å in this example.

In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific, it is to be understood that inventors appreciate and understand that any and all data points within the range are to be considered to have been specified, and that inventors possessed knowledge of the entire range and all points within the range.

As used in the specification and claims, “near” is inclusive of “at.” The term “and/or” refers to both the inclusive “and” case and the exclusive “or” case, and such term is used herein for brevity. For example, a composition comprising “A and/or B” may comprise A alone, B alone, or both A and B.

Various components described in this specification may be described as “including” and/or made of, and/or “having” certain materials, properties, or compositions of material(s). In one aspect, this can mean that the component consists of certain materials, properties, or compositions of materials. In another aspect, this can mean that the component comprises certain materials, properties, or compositions of material(s).

The word “exemplary” is used herein to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another-even if they do not directly physically touch each other.

In the disclosure various ranges in values may be specified, described and/or claimed. It is noted that any time a range is specified, described and/or claimed in the specification and/or claim, it is meant to include the endpoints (at least in one aspect).

In another aspect, the range may not include the endpoints of the range. In the disclosure various values (e.g., value X) may be specified, described and/or claimed. In one aspect, it should be understood that the value X may be exactly equal to X. In one aspect, it should be understood that the value X may be “about X,” with the meaning noted above. Likewise, when a value is determined according to an equation, it is to be understood that in one aspect, the value is equal to the value calculated according to the equation and in another aspect, the value is about equal to the value calculated according to the equation according to the meaning noted above, or as is expressly provided for, e.g., plus or minus (+/−) a specific amount.

While various aspects have been described above, it should be understood that they have been presented by way of example only, and not limitation. Thus, the breadth and scope of an aspect of the present disclosure should not be limited by any of the above-described exemplary aspects, but should be defined only in accordance with the following claims and their equivalents.

All documents described herein are incorporated by reference herein, including any priority documents and/or testing procedures to the extent they are not inconsistent with this text. As is apparent from the foregoing general description and the specific embodiments, while forms according to various aspects of the disclosure have been illustrated and described, various modifications can be made without departing from the spirit and scope of the disclosure. Accordingly, it is not intended that the invention be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

1. A composition comprising a plurality of discrete particles comprising one or more cannabinoids disposed at least partially within a polymeric carrier having a maximum overall dimension of less than 1 micron.

2. The composition of claim 1, produced by electrospray of a solution comprising one or more cannabinoids and the polymeric carrier.

3. The composition of claim 1, produced by coaxial electrospray including an outer flow comprising the polymeric carrier, and an inner flow comprising the one or more cannabinoids.

4. The composition of claim 1, wherein the one or more cannabinoids include a tetrahydrocannabinol (THC), a cannabidiol (CBD), a cannabivarin (CBV), a tetrahydrocannabivarin (THCV), or a combination thereof.

5. The composition of claim 1, wherein the polymeric carrier includes a gelatin, ethyl cellulose, or a combination thereof.

6. The composition of claim 1, comprising greater than or equal to about 30 wt % of the one or more cannabinoids.

7. The composition of claim 1, wherein a 10 wt % mixture of the composition in water at 25° C. forms a clear solution.

8. A composition comprising a plurality of discrete nanofibers comprising one or more cannabinoids disposed at least partially within a polymeric carrier having a maximum width of less than 1 micron.

9. The composition of claim 8, produced by electrospinning of a solution comprising one or more cannabinoids and the polymeric carrier.

10. The composition of claim 8, produced by coaxial electrospinning including an outer flow comprising the polymeric carrier, and an inner flow comprising the one or more cannabinoids.

11. The composition of claim 8, wherein the one or more cannabinoids include a tetrahydrocannabinol (THC), a cannabidiol (CBD), a cannabivarin (CBV), a tetrahydrocannabivarin (THCV), or a combination thereof.

12. The composition of claim 8, wherein the polymeric carrier includes a gelatin, ethyl cellulose, or a combination thereof.

13. The composition of claim 8, comprising greater than or equal to about 30 wt % of the one or more cannabinoids.

14. The composition of claim 8, wherein a 10 wt % mixture of the composition in water at 25° C. forms a clear solution.

15. A process to produce a composition comprising the steps of:
a) providing one or more precursor mixtures comprising one or more cannabinoids and one or more polymeric carrier components in a solvent; and
i) electrospraying these one or more precursor mixtures under electrospray conditions to form a composition including a plurality of discrete particles comprising one or more cannabinoids at least partially encapsulated with or disposed on the polymeric carrier, each of said discrete particles having a maximum dimension of less than or equal to about 1 micron, and/or agglomerates of said discrete particles; or
ii) electrospinning these one or more precursor mixtures under electrospinning conditions to form a composition including a plurality of discrete nanofibers comprising one or more cannabinoids at least partially encapsulated with or disposed on the polymeric carrier, each of said discrete nanofibers having a maximum width of less than or equal to about 1 micron.

16. The process according to claim 15, wherein a first precursor mixture comprises one or more cannabinoids in a solvent;
a second precursor mixture comprises one or more polymeric carrier components dissolved and/or dispersed in a solvent; and
each of the precursor mixtures are coaxially electrosprayed to form a plurality of discrete particles comprising one or more cannabinoids at least partially encapsulated with or disposed on the polymeric carrier, each of said discrete particles having a maximum dimension of less than or equal to about 1 micron.

17. The process according to claim 16, wherein the one or more cannabinoids include a tetrahydrocannabinol (THC), a cannabidiol (CBD), a cannabivarin (CBV), a tetrahydrocannabivarin (THCV), or a combination thereof.

18. The process according to claim 15, wherein a first precursor mixture comprises one or more cannabinoids in a solvent;
a second precursor mixture comprises one or more polymeric carrier components dissolved and/or dispersed in a solvent; and
each of the precursor mixtures are coaxially electrospun to form a plurality of discrete nanofibers comprising one or more cannabinoids at least partially encapsulated with or disposed on the polymeric carrier, each of said discrete nanofibers having a maximum width of less than or equal to about 1 micron.

19. The process according to claim 18, wherein the one or more cannabinoids include a tetrahydrocannabinol (THC), a cannabidiol (CBD), a cannabivarin (CBV), a tetrahydrocannabivarin (THCV), or a combination thereof.

20. The process of claim 15, wherein the composition comprises greater than or equal to about 30 wt % of the one or more cannabinoids.
